(19) United States
(12) Patent Application Publication
(10) Pub. No.: US 2025/0387510 A1
(43) Pub. Date: Dec. 25, 2025

(54) ADENO-ASSOCIATED VIRUS FORMULATIONS

(71) Applicant: Spark Therapeutics, Inc., Philadelphia, PA (US)

(72) Inventors: Smrithi Padmakumar, Philadelphia, PA (US); Vineet Kumar, Chester Springs, PA (US); Matthew Petroff, Philadelphia, PA (US); Karen Collette Baker, Tempe, AZ (US); Leslie Lam, Richboro, PA (US)

(73) Assignee: Spark Therapeutics, Inc., Philadelphia, PA (US)

(21) Appl. No.: 19/246,483

(22) Filed: Jun. 23, 2025

Related U.S. Application Data
(63) Continuation of application No. PCT/US2023/085699, filed on Dec. 22, 2023.
(60) Provisional application No. 63/477,017, filed on Dec. 23, 2022.
(60) Provisional application No. 63/612,443, filed on Dec. 20, 2023.

(51) Int. Cl. A61K 48/00; A61K 9/19; A61K 47/02; A61K 47/10; A61K 47/12; A61K 47/26; C07K 14/005; C12N 7/00; C12N 15/86

(52) U.S. Cl. CPC A61K 48/0041; A61K 9/19; A61K 47/02; A61K 47/10; A61K 47/12; A61K 47/26; C07K 14/005; C12N 7/00; C12N 15/86; C12N 2750/14122; C12N 2750/14143

(57) ABSTRACT

Compositions and methods for the formulation and use of recombinant adeno-associated viruses (rAAVs) are described. In certain embodiments, the application discloses recombinant Adeno-Associated Virus (rAAV or AAV) formulations that provide for one or more of the following: maintain stable freeze-thaw and lyophilization (freeze-drying) performance, and enable longer term shelf storage at temperatures above-80° C. while maintaining critical quality attributes such as genome recovery, retention of potency, minimal aggregation and degradation, improved vector quality, retaining viral protein (VP) ratios, maximum chemical stability (minimal deamidation and oxidation) and/or improved thermodynamic stability.

### FIELD OF INVENTION

The application relates to compositions and methods for the formulation and use of gene therapy drug products. In certain embodiments, the application discloses recombinant Adeno-Associated Virus (rAAV or AAV) formulations that provide for one or more of the following: maintain stable freeze-thaw and lyophilization (freeze-drying) performance, and enable longer term shelf storage at temperatures above −80° C. while maintaining critical quality attributes such as genome recovery, retention of potency, minimal aggregation and degradation, improved vector quality, retaining viral protein (VP) ratios, maximum chemical stability (minimal deamidation and oxidation) and/or improved thermodynamic stability.

### CROSS-REFERENCE TO RELATED APPLICATIONS

The present application is a continuation of International Application No. PCT/US2023/085699, filed Dec. 22, 2023, which claims priority to U.S. Provisional Application No. 63/477,017, filed Dec. 23, 2022, and U.S. Provisional Application No. 63/612,443, filed Dec. 20, 2023.

### REFERENCE TO SEQUENCE LISTING SUBMITTED ELECTRONICALLY

The contents of the electronic sequence listing (089504.0357US.xml; Size: 5,457 bytes; and Date of Creation: Jun. 20, 2025) is herein incorporated by reference in its entirety.

### BACKGROUND

Challenges to rAAV formulation stability include protein degradation, aggregation and loss of efficacy. Significant formulation degradation pathways include (1) freeze/thaw induced unfolding and activity loss; and (2) aggregation at low ionic strength. Potential degradation mechanisms include physical instability, aggregation, surface adsorption, chemical instability, disulfide formation/exchange, deamidation, oxidation, and isomerization (Srivastavaz et al., Journal of Pharmaceutical Sciences, volume 110, issue 7, 2021, pages 2609-2624.)

Current rAAV-based therapeutics are generally formulated for frozen storage, e.g., storage at −80° C.±10° C., and such formulations include ingredients, e.g., certain salts, phosphate buffer species, and surfactants, at concentrations to provide osmolarity compatible with expected routes of administration, to prevent aggregation, enhance stability, and minimize loss to product surfaces. These formulations, however, may exhibit unpredictable performance under freeze-thaw conditions, for example, due to their inclusion of phosphate buffer-based matrices, which can also lead to pH shifts. In addition, the lack of a sugar component in conventional formulations hinders their amenability for lyophilization, which is important for targeting higher shelf storage temperatures, e.g., 2-8° C.

One particular challenge in the formulation of rAAV therapies is the high ionic strength formulations common within the rAAV art (often with NaCl concentration of 150 mM or higher), creating high baseline osmolality, low glass transition temperature (T_{g}′) and limiting inclusion of additional excipients such as sugars.

International Patent Publication Nos. WO2020214929 and WO2018128689 include examples of rAAV formulations.

In view of the foregoing, there is a need in the art for new stable rAAV formulations, having one or more of the following properties: amenable to lyophilization, capable of maintaining predictable freeze-thaw performance, exhibit enhanced long-term shelf stability at higher temperatures in various forms, e.g., frozen, liquid or lyophilized, and are compatible with various routes of administration including systemic, ocular, and CNS.

### SUMMARY OF THE INVENTION

In certain embodiments, the application is directed to compositions and methods for the formulation and use of gene therapy products. In particular, the application relates to rAAV formulations that are amenable to at least one of, or all of, lyophilization, maintain predictable freeze-thaw performance, and exhibit enhanced long-term stability at −80° C., −40° C., −20° C., 2-8° C. and 25° C.

In one general aspect, the application relates to a stable formulation comprising a therapeutic drug product and:
(a) 1-100 mM one or more salts;
(b) a buffering agent;
(c) 0.001-0.05% by weight one or more non-ionic surfactants;
(d) 3-10% by weight one or more sugars; and
(e) water,
(f) optionally, one or more amino acids, and
   wherein the composition has a pH of 4.5-7.5, and 60% or more of the drug product is recovered after the formulation is frozen and thawed.

In another general aspect, the application relates to a stable formulation comprising a therapeutic drug product and:
(a) 1-100 mM one or more salts;
(b) a buffering agent;
(c) 0.001-0.05% by weight one or more non-ionic surfactants;
(d) 3-10% by weight one or more sugars;
(e) water; and
(f) optionally, one or more amino acids, and
   wherein the composition has a pH of 4.5-7.5, and 60% or more of the drug product is recovered after the formulation is lyophilized.

Another general aspect of the application relates to a stable formulation comprising:
(a) a recombinant adeno-associated virus (AAV);
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5.

Another general aspect of the application relates to a stable formulation comprising:
(a) a recombinant adeno-associated virus (AAV);
(b) 10 mM Tris (Tris(hydroxymethyl)aminomethane);
(c) 75 mM sodium chloride;
(e) 0.005% by weight poloxamer 188;
(d) 5% by weight sucrose;
(e) water; and
(f) at a pH of 7.3.

In another general aspect, the application relates to a stable formulation comprising:
(a) a recombinant adeno-associated virus (rAAV);
(b) 10 mM sodium citrate;
(c) 75 mM sodium chloride;
(d) 0.001% by weight poloxamer 188;
(e) 7% by weight sucrose;
(f) water; and
(g) at a pH of 6.0.

Another general aspect of the application relates to a method of reducing degradation of a therapeutic drug product after freeze-thawing said product, the method comprising preparing a composition comprising:
(a) the therapeutic drug product;
(b) 1-100 mM one or more salts;
(c) a buffering agent;
(d) 0.001-0.05% by weight one or more non-ionic surfactants;
(e) 3-10% by weight one or more sugars; and
(f) water,
(g) at a pH of 4.5 to 7.5; and
(h) optionally, one or more amino acids, and
   freezing said composition for a duration at a temperature above −80° C. and up to −10° C., such as a temperature of −70° C. to −15° C., −60° C. to −20° C., −50° C. to −30° C., or −45° C. to −35° C., wherein 60% or more of the therapeutic drug product is recovered after thawing compared to the same product stored at −80° C. for same duration.

Another general aspect of the application relates to a method of reducing degradation of a therapeutic drug product after freeze-thawing said product, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein 60% or more of the therapeutic drug product is recovered after thawing compared to the same product stored at −80° C. for same duration.

Another general aspect of the application relates to a method of retaining at least 50% relative potency of a therapeutic drug product after freeze-thawing said product, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −60° C. and up to −10° C., such as −60° C. to −10° C., −50° C. to −20° C., −45° C. to −25° C., or −30° C. to −40° C., wherein after thawing, said drug product exhibits at least 50% relative potency compared to the same product stored at −80° C. for the same duration.

Another general aspect of the application relates to a method of reducing deamination of amino acids on capsid of an rAAV, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5.

Another general aspect of the application relates to a method of reducing degradation of a therapeutic drug product after lyophilizing said product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water;
(g) at a pH of 7.3; and
   storing said lyophilized composition for a duration at a temperature between −20° C. and 8° C., such as a temperature of −20° C. to 0° C. or 0° C. to 8° C., wherein 60% or more of the therapeutic drug product is recovered after reconstituting lyophilized product compared to the same product stored at −80° C. without lyophilization.

Another general aspect of the application relates to a method of retaining at least 50% relative potency of a therapeutic drug product after lyophilizing said product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water; and
(g) at a pH of 7.3; and
   storing said lyophilized composition for a duration at a temperature between −20° C. and 8° C., such as a temperature of −20° C. to 0° C. or 0° C. to 8° C., wherein said drug product exhibits at least 50% relative potency after reconstituting lyophilized product compared to the same product stored at −80° C. without lyophilization.

Another general aspect of the application relates to a method of reducing deamination of amino acids on capsid of a rAAV, the method comprising preparing a composition comprising:
(a) the rAAV;
(b) 10 mM Tris;
(c) 75 mM sodium chloride;
(d) 0.005% by weight poloxamer 188;
(e) 5% by weight sucrose;
(f) water; and
(g) at a pH of 7.3.

Another general aspect of the application relates to a method of reducing degradation of a therapeutic drug product after lyophilizing said product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein 60% or more of the therapeutic drug product is recovered after reconstituting lyophilized product compared to the same product stored at −80° C. without lyophilization.

Another general aspect of the application relates to a method of retaining at least 50% relative potency of a therapeutic drug product after lyophilizing said product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein said drug product exhibits at least 50% relative potency after reconstituting lyophilized product compared to the same product stored at −80° C. without lyophilization.

Another general aspect of the application relates to a method of reducing deamination of amino acids on capsid of an rAAV, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose;
(f) water; and
(g) at a pH of 6.0.

Another general aspect of the application relates to a method of reducing low molecular weight species (LMW) after lyophilizing the product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water; and
(g) at a pH of 7.3; and
   storing said lyophilized composition for a duration at a temperature between −20° C. and 8° C., such as a temperature of −20° C. to 0° C. or 0° C. to 8° C., wherein the lyophilized product is reconstituted and LMW species are less than 5% by weight compared to the same product without lyophilization as measured by analytical ultracentrifugation.

Another general aspect of the application relates to a method of reducing low molecular weight species (LMW) after lyophilizing the product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein the lyophilized product is reconstituted and LMW species are less than 5% by weight compared to the same product without lyophilization as measured by analytical ultracentrifugation.

Another general aspect of the application relates to a method of reducing degradation of viral proteins after freezing the product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5; and
   storing said frozen composition for a duration at a temperature between −80° C. and up to −10° C., such as a temperature of −70° C. to −15° C., −60° C. to −20° C., −50° C. to −30° C., or −45° C. to −35° C., wherein the change in the ratio VP1: VP3 is less than 20% after thawing the frozen product.

Another general aspect of the application relates to a method of reducing degradation of viral proteins after lyophilizing the product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water; and
(g) at a pH of 7.3; and
   storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to the sample before lyophilization.

Another general aspect of the application relates to a method of reducing degradation of viral proteins after lyophilizing the product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to the sample before lyophilization.

Another general aspect of the application relates to a method of reducing subvisible particle (SVP) concentration of a therapeutic drug product after freeze-thawing said product, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for at least one year at −20° C., wherein after thawing, SVP concentration is less than 100 particles per mL for particles 10 μm or larger, and less than 10 particles per mL for particles 25 μm or larger.

Another general aspect of the application relates to a method of reducing subvisible particle (SVP) concentration of a therapeutic drug product after 5 freeze-thaw cycles, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein said composition is thawed and refrozen five times, and upon the fifth thaw, SVP concentration is less than 100 particles per mL for particles 10 μm or larger, and less than 10 particles per mL for particles 25 μm or larger.

Another general aspect of the application relates to a method of reducing subvisible particle (SVP) concentration of a therapeutic drug product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water; and
(g) at a pH of 7.3; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 100 particles per mL for particles 10 μm or larger, and less than 10 particles per mL for particles 25 μm or larger.

Another general aspect of the application relates to a method of reducing subvisible particle (SVP) concentration of a therapeutic drug product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 100 particles per mL for particles 10 μm or larger, and less than 10 particles per mL for particles 25 μm or larger.

According to embodiments of the application, the therapeutic drug product is a rAAV. The rAAV can comprise a capsid derived from one or more AAVs selected from the group consisting of AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1, DJ, DJ8, NP59, Anc-80, and variants thereof. The rAAV can comprise a transgene that encodes a polypeptide, or a nucleic acid selected from the group consisting of a siRNA, an antisense molecule, miRNA, a ribozyme and a shRNA.

According to embodiments of the application, the rAAV used in an embodiment of the application comprises the transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USHIC (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1 depicts the vector recoveries for each of the formulations at all temperatures (5° C., −20° C., −40° C., −80° C.) and time points tested (2 weeks-6 months) calculated as a percentage of the T0 titers. F1 (Formulation 1)=25 mM Na Acetate, 25 mM NaCl, 5 mM MgCl₂, pH 5.2, 5.4% Sucrose, 0.01% Pluronic™ F-68 (Poloxamer 188); F2 (Formulation 2)=10 mM Na Phosphate, 180 mM NaCl, pH 7.3, 0.001% Poloxamer 188; and F3 (Formulation 3)=10 m M Na Phosphate, 60 mM NaCl, 3% Sucrose, pH 7.3, 0.001% Poloxamer 188.

FIG. 2 depicts the IEX peak retention times for each of the formulations at all temperatures (5° C., −20° C., −40° C., −80° C.) and time points tested (2 weeks-6 months).

FIG. 3 depicts the Total High Molecular Weight (HMW) species for all samples reported by SEC-MALS (size exclusion chromatography-multi angle light scattering) analyses for each of the formulations at all temperatures (5° C., −20° C., −40° C., −80° C.) and time points tested (2 weeks-6 months).

FIG. 4 depicts the VP capsid protein ratios for all samples reported by CE-SDS (capillary electrophoresis with sodium dodecyl sulfate) for each of the formulations at all temperatures (5° C., −20° C., −40° C., −80° C.) and time points tested (2 weeks-6 months).

FIG. 5 depicts fluorescence intensity data and Tm (melting point) of all T0 samples from intrinsic differential scanning fluorimetry (DSF). 1 and 2 indicates replicate measurements within each formulation samples. The dotted arrow shows the peak shift seen for F1.

FIG. 6 depicts extrinsic DSF fluorescence intensities normalized for different T0 formulations.

FIG. 7 depicts High Accuracy (HIAC) light obscuration measurements of samples showing subvisible particle numbers for each of the formulations at all temperatures (5° C., −20° C., −40° C., −80° C.) and time points tested (2 weeks-6 months).

FIG. 8 depicts peptide mapping data showing the levels of deamidation and oxidation in the hotspot residues for each of the formulations at the specified temperatures and time points tested.

FIG. 9 depicts relative potency values of all samples relative to T0 samples for each of the formulations at all temperatures (5° C., −20° C., −40° C., −80° C.) and time points tested (2 months and 6 months).

FIG. 10 depicts analysis of matrix conditions that affect rAAV titer during liquid stress at 30° C. Preferred conditions have increase % remaining after stress, and are at the combination of pH<7.3, NaCl<100 mM, MgCl2<10 mM.

FIGS. 11A-11D depict the effect of certain buffer and excipients on subvisible particulate (SVP) formation during rAAV freeze-thaw. FIGS. 11A-11B illustrate the significant dependence SVP number and size have on buffering species, freeze-thaw rate, and fraction of rAAV particles without therapeutic genome (i.e. “empty” rAAV fraction). FIGS. 11C-11D illustrate optimized storage conditions (i.e. presence of sugar and surfactant as cryoprotectants) with significant reduction in SVP formation and reduced SVP formation on buffering species, freeze-thaw rate, and empty rAAV fraction.

FIG. 12 depicts the pH of rAAV in formulation F1, measured in the thawed solution after long term storage.

FIGS. 13A and B depict the titer of rAAV in formulation F1 during long term storage. Titer of intact viral genomes measured by droplet digital PCR after each time and temperature combination. FIG. 13A depicts the total vector genome recovered. FIG. 13B depicts titer normalized by the initial measured titer (T0).

FIG. 14 depicts the cell-based potency of rAAV in formulation F1 during long term storage. In-vitro potency measured by cell-based functional assay. All measurements are relative to the potency measured before long-term stability (T0 condition).

FIGS. 15A and B depict the cell-based infectivity of rAAV in formulation F1 during long term storage. Infectivity is measured by cell-based assay for each time and temperature combination. FIG. 15A depicts vector-particle concentration to infectious vector titer ratio, or PI ratio. FIG. 15B depicts infectious titers.

FIGS. 16A and B depict the total capsids and empty: full ratio of rAAV in formulation F1 measured by absorbance and 280 nm and 260 nm, during long term storage. FIG. 16A depicts the total capsid concentration. FIG. 16B depicts the ratio of empty: full viral capsids.

FIGS. 17A, B, and C depict the ratio of capsid viral proteins of rAAV in formulation F1 during long-term storage. FIG. 17A depicts the ratio of viral proteins normalized to viral protein 1 (VP1), including viral protein 2 (VP2), viral protein 3 (VP3), and a VP 3 variant termed Pre VP3. FIG. 17B depicts the fraction of mass attributed to the combination of VP1, VP2, and VP3. FIG. 17C depicts the fraction of protein attributed to degradant species that are not VP1, VP2, or VP3.

FIG. 18 depicts oligomeric high-molecular weight rAAV impurities in in formulation F1 during long term storage. Impurities are measured by analytical size exclusion chromatography, and reported as the percent area by absorbance at 280 nm.

FIG. 19 depicts rAAV capsid deamidation in formulation F1 during long term storage, reported for key amino acids N57, N254, N255, N263, and N514. Deamidation was measured with peptide mapping mass spectrometry.

FIG. 20 depicts percent recovery of reconstituted rAAV after a lyophilization cycle. Recovery is of the viral genome by qPCR, normalized by the titer before lyophilization. rAAV are lyophilized in two formulations, F4 and F5, defined below, with secondary drying (SD) at either 25° C. or 30° C. F4 (Formulation 4)=10 mM Tris, 75 mM NaCl, 5% Sucrose, 0.005% Poloxamer 188, pH 7.3; F5 (Formulation 5)=10 mM sodium citrate, 75 mM NaCl, 7% sucrose, 0.001% Poloxamer, pH 6.0.

FIG. 21 depicts cell-based potency of reconstituted rAAV after a lyophilization cycle. Potency is normalized to the sample before lyophilization. rAAV is lyophilized in formulation F4, with secondary drying at either 25° C. or 30° C.

FIG. 22 depicts oligomeric high-molecular weight species in reconstituted rAAV solutions after lyophilization, measured by SEC-MALS. rAAV are lyophilized in formulations F4 and F5 formulations, with secondary drying (SD) at either 25° C. or 30° C. T0 filtered indicates 0.2 μm filtered samples before lyophilization.

FIG. 23 depicts the ratio of capsid proteins (VP1, VP2, VP3, and pre-VP3 variant) in reconstituted rAAV solution after lyophilization. Viral protein abundance is measured by CE-SDS, and normalized to the amount of VP1 in each sample. T0 indicates value before lyophilization.

FIG. 24 depicts the titer recovery of reconstituted rAAV during long term storage and relative humidity (RH). rAAV is lyophilized in F4, and stored between −20° C. and 35° C. for up to 6 months. Titer is measured by qPCR, and recovery is calculated by the titer before lyophilization (T0 liquid).

FIG. 25 depicts the cell-based potency of reconstituted rAAV. rAAV is lyophilized in F4, and stored between −20° C. and 35° C. for up to 6 months. Potency is normalized to the potency before lyophilization (T0 liquid).

FIGS. 26A and B depict the fraction of oligomeric high molecular weight (HMW) in reconstituted rAAV, assessed by SEC-MALS. rAAV is lyophilized in F4, and stored between −20° C. and 35° C. for up to 6 months. FIG. 26A illustrates the percent monomer. FIG. 26B illustrates the percent HMW.

FIG. 27 depicts the fraction of high molecular weight (HMW) in reconstituted rAAV, assessed by analytical ultracentrifugation, and reported for changes in HMW, partial capsids (partial) and low molecular weight (LMH) species. rAAV is lyophilized in F4, and stored between-20° C. and 35° C. for up to 6 months.

FIG. 28 depicts the concentration of subvisible particulate in reconstituted rAAV, assessed by light obscuration (HIAC). rAAV is lyophilized in F4, and stored between −20° C. and 35° C. for up to 6 months. Particulate concentration is in size bins of ≥5 um, ≥10 μm, and ≥ 25 um. T0 liquid indicates sample prior to lyophilization, and 0 Day Post Lyo indicates sample after lyophilization cycle sample.

FIG. 29 depicts the ratio of rAAV capsid proteins (VP1, VP2, VP3, and pre-VP variant) in reconstituted solution. rAAV is lyophilized in F4, and stored between −20° C. and 35° C. for up to 6 months. Viral protein abundance is measured by CE-SDS, and normalized to the amount of VP1 in each sample. T0 liquid indicates sample before lyophilization.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

In certain embodiments, the present disclosure is directed to compositions and methods for the formulation and use of gene therapy products. In particular, the application discloses stable rAAV formulations preferably having one or more of the following properties: maintain predictable (stable) freeze-thaw and lyophilization (freeze-drying) performance, and enable longer term shelf storage at temperatures above −60° C. while maintaining critical quality attributes such as genome recovery, retention of potency, minimal aggregation and degradation, improved vector quality, maximal chemical stability (e.g., minimal deamidation and oxidation) and/or maximal thermodynamic stability.

In certain embodiments, an expected stability time period is at least 6 months, preferably 1 year, and most preferably 2 years. If an accelerated condition (usually a significantly higher or lower temp than normal storage conditions) shows acceptable changes within the timeframe of observation, it is reasonable to extrapolate to the stability results to the next time period of observation (typically intervals of 6 to 12, 18 to 24, 36 to 48, etc.). For frozen storage conditions without accelerated stability conditions, if there is no change within the current timeframe, then it is expected the next timeframe will also be acceptable. (Compendial guidance; European Medicines Agency, Note For Guidance On Evaluation Of Stability Data, CPMP/ICH/420/02, August 2003; and USP <787>).

Unless defined otherwise, all technical and scientific terms used herein generally have their ordinary meanings in the art to which this invention pertains, within the context of this disclosure and in the specific context where each term is used. Certain terms are discussed below, or elsewhere in the specification, to provide additional guidance to the practitioner in describing the compositions and methods of the present disclosure and how to make and use them.

As used herein and in the appended claims, the singular forms “a,” “an,” and “the” include plural reference unless the context clearly dictates otherwise. For example, the use of the word “a” or “an” when used in the claims and/or the specification can mean “one,” but it is also consistent with the meaning of “one or more,” “at least one” and “one or more than one.”

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element, where such element, step or ingredient is related to the claimed invention. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

All of the features disclosed herein can be combined in any combination. Each feature disclosed in the specification can be replaced by an alternative feature serving a same, equivalent, or similar purpose.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. With respect to formulation components and pH, “about” indicates plus or minus 10% of the indicated value.

The term “vector” refers to carrier nucleic acid molecule that can be manipulated by insertion or incorporation of a nucleic acid. Examples of “vector” include, but are not limited to, a plasmid, a virus, including an rAAV vector, or other vehicle capable of delivering a nucleic acid molecule. Vectors can be used for genetic manipulation to introduce or transfer polynucleotides into cells, and to transcribe or translate the inserted polynucleotide in cells. An “expression vector” is a specialized vector that contains a gene or nucleic acid sequence with the necessary regulatory regions needed for expression in a host cell.

A viral vector is derived from or based upon one or more nucleic acid elements that comprise a viral genome and may also comprise a viral caspid encapsidating the nucleic acid. A particular viral vector is an adeno-associated virus (AAV) vector, referred to herein as “rAAV”.

The term “recombinant,” as a modifier of a composition, means that the composition has been manipulated or engineered in a fashion that generally does not occur in nature. A recombinant composition includes, a recombinant vector, such as recombinant AAV vector, a recombinant polynucleotide or polypeptide, or a recombinant cell or animal. The term “recombinant,” as a modifier of nucleic acid or a vector indicates a combination of elements that does not occur in nature. Examples of recombinant nucleic acid include a recombinant viral vector nucleic acid providing 5′ and/or 3′ viral elements along with an expression cassette containing one or more elements not naturally associated with the 5′ and/or 3′ elements; and expression cassettes that may contain different recombined components such a heterologous protomer, polyA, introns and spacers. Similarly, a viral vector, such as an rAAV vector may contain a naturally occurring or modified capsid, encapsidating recombinant viral vector nucleic acid. A particular example of a recombinant AAV vector can be an rAAV vector where a nucleic acid sequence that is not normally present in the wild-type AAV genome (e.g., a heterologous nucleic acid sequence) is inserted within the AAV genome. Although the term “recombinant” is not always used herein in reference to rAAV vectors, as well as sequences such as polynucleotides, recombinant forms of the rAAV vector and polynucleotides are expressly included in spite of any such omission.

Viral vector nucleic acid contain 5′ and/or 3′ viral elements providing for viral packaging and may provide for additional activities such as self-priming, DNA replication, promoter activity, genome integration, or episomal concatermerization. The 5′ and 3′ elements are generally located at or near the 5′ and 3′ terminal end of the recombinant viral vector nucleic acid and can be naturally occurring or modified versions of naturally occurring sequences. (Naso et al., (2017) BioDrugs, 31 (4), 317-334; and Bulcha et al., (2021) Sig. Transduct. Target Ther. 6:53 (2021).

Additional elements for rAAV vectors include, without limitation, a transcription termination signal or stop codon, 5′ or 3′ untranslated regions (e.g., polyadenylation (polyA) sequences) which flank a sequence, such as one or more copies of an AAV ITR sequence, or an intron. Nucleic acid elements include expression vector components comprising a transgene along with regulatory elements providing for and/or facilitating translation, nuclear export of nucleic acid, and translations. Further elements include, for example, filler or stuffer polynucleotide sequences, for example to improve packaging and reduce the presence of contaminating nucleic acid. For shorter sequences, inclusion of a stuffer or filler sequence may be used to adjust the length of the total nucleic acid sequence to the size of the virus' genomic sequence acceptable for AAV vector packaging into virus particle. In various embodiments, a filler or stuffer nucleic acid sequence is an untranslated (non-protein encoding) segment of nucleic acid.

Where a wild type heterologous nucleic acid or transgene is too large to be packaged within an AAV vector particle, and AAV delivery is desired, the heterologous nucleic acid may be provided in modified, fragmented or truncated form for packaging in and delivery by an AAV vector, such that a functional protein or nucleic acid product, such as a therapeutic protein or nucleic acid product, is ultimately provided.

Recombinant AAV vector nucleic acid can be derived from a wild type (wt or wild-type) genome of AAV by using molecular methods to modify the wild type genome. For example, a rAAV can be obtained by removing a portion of native nucleic acid sequence from the AAV genome, and replacing it with a non-native nucleic acid sequence, referred to as a heterologous nucleic acid. Alternatively, wild-type ITR or variants thereof, can be appended 3′ and/or 5′ to a heterologous sequence. Typically, one or both inverted terminal repeat (ITR) sequences of AAV genome are retained in the rAAV vector. rAAV is distinguished from an AAV genome, since in rAAV, all or a part of the AAV genome has been replaced or otherwise modified with a non-native sequence with respect to the AAV genomic nucleic acid. Incorporation of a non-native sequence therefore defines the AAV vector as a “recombinant” vector, which can be referred to as a “rAAV vector.”

Reference to a viral vector provides for viral nucleic acid and can also provide for a capsid (also referred to as a particle). The term “rAAV” can be used to refer recombinant viral vector nucleic acid and/or recombinant viral vector nucleic acid encapsidated in a capsid. Viral vectors can be used, for example, for infection (transduction) of a cell, ex vivo, in vitro or in vivo.

A vector “genome” refers to the portion of the recombinant plasmid sequence that is ultimately packaged or encapsidated to form a viral (e.g., rAAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, but is not itself packaged or encapsidated into virus (e.g., rAAV) particles. Thus, a vector “genome” refers to the nucleic acid that is packaged or encapsidated by virus (e.g., the nucleic acid packaged in an rAAV).

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA. Inhibitory RNA includes, but is not limited to, small or short hairpin (sh) RNA, microRNA (miRNA), small or short interfering (si) RNA, trans-splicing RNA, or antisense RNA. Nucleic acids include naturally occurring, synthetic, and intentionally modified or altered polynucleotides (e.g., variant nucleic acid). The nucleic acids include, but are not limited to, cDNA, genomic DNA, RNA, and fragments thereof which may be single- or double-stranded.

Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

A viral protein (VP) as used herein is at least one of the three subunit proteins (any variants thereof) that comprise the AAV capsid, referred to as viral protein 1, viral protein 2, and viral protein 3 (VP1, VP2, VP3), and pre-VP3. Reducing the degradation of viral peptide proteins is typically observed by a decrease in the relative abundance of VP1 and/or VP2 compared to VP3. The degradation of viral proteins can be presented as an increase in the relative amount of VP3 (and similar species including pre-VP3) compared to VP1 or VP2. One non-limiting example is to determine, before and after a storage condition, the ratio of VP1: VP3, where VP3 includes pre-VP3 (e.g. VP3+preVP3), and then compare. Degradation is present if the ratio of VP1: VP3 is higher after storage. To avoid confusion, in the present application, when VP3 is calculated in determining degradation, the value is VP3+preVP3.

A “transgene” is used herein to conveniently refer to a heterologous nucleic acid that is intended or has been introduced into a cell or organism. Transgenes include any heterologous nucleic acid that encodes a polypeptide or protein or encodes an inhibitory RNA, that is included in a vector, particularly an rAAV. Transgenes can directly increase levels of a target protein by encoding for the RNA that the host cell machinery processes and translates into the target protein. Transgenes can indirectly increase levels of a target protein by either encoding for regulatory proteins that promote target protein expression or by encoding RNA that inhibits proteins and/or nucleic acids that serve to repress the target protein. A transgene can be a gene of interest (GOI) as described below.

A heterologous nucleic acid can be introduced or transferred by way of vector, such as rAAV, “transduction” or “transfection” into a cell. The term “transduce” and grammatical variations thereof refer to introduction of the vehicle carrying the nucleic acid into a cell or host organism. The introduced heterologous nucleic acid may also exist in the recipient cell or host organism extrachromosomally, or only transiently.

A “transduced cell” is a cell into which the transgene or heterologous nucleic acid has been introduced. Accordingly, a “transduced” cell means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced nucleic acid transcribed, and if the nucleic acid encodes a protein, subsequent translation of the nucleic acid transcript. For gene therapy uses and methods, a transduced cell can be in a subject.

An “expression control element” is a type of regulatory element and refers to nucleic acid sequence(s) that influence expression of an operably linked nucleic acid. Typically, such elements are included to facilitate proper heterologous polynucleotide transcription, and, if appropriate, translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but can also act in trans.

The term “operably linked” means that the regulatory sequences modulates expression of a nucleic acid sequence are placed in the appropriate positions relative to the sequence so as to effect expression of the nucleic acid sequence. This same definition is sometimes applied to the arrangement of nucleic acid sequences and transcription control elements (e.g., promoters, enhancers, and termination elements) in an expression vector, e.g., rAAV vector.

As used herein, an “E: F ratio” or “empty AAV fraction” refers to the ratio of the empty rAAV particles not packaged with an rAAV genome to full rAAV particles packaged with an rAAV genome.

“Degradation” refers to the general set of chemical and physical changes that accumulate over time, degrading the safety and/or efficacy of the vector. These may include, but are not limited to, ejection of genome, aggregation of vector, fragmentation of capsid proteins, and deamidation of capsid proteins. Further, in some embodiments of the invention, degradants are referred to as high molecular weight species (HMW) or low molecular weight species (LMW).

In some embodiments, the heterologous nucleic acid that encodes a protein (e.g., therapeutic protein) is provided in modified or truncated forms or the heterologous nucleic acid is provided in multiple constructs, delivered by separate and multiple AAV vectors.

In certain aspects, the heterologous nucleic acid is provided as a truncated variant that maintains functionality of the encoded protein (e.g., therapeutic protein), including removal of portions unnecessary for function, such that the encoding heterologous polynucleotide is reduced in size for packaging in an AAV vector.

In certain aspects the heterologous nucleic acid is provided in split AAV vectors, each providing nucleic acid encoding different portions of a protein (e.g., therapeutic protein), thus delivering multiple portions of a protein (e.g., therapeutic protein) which assemble and function in the cell.

In other aspects, the heterologous nucleic acid is provided by dual AAV vectors using overlapping, trans-splicing or hybrid trans-splicing dual vector technology. In certain embodiments, two overlapping AAV vectors are used which combine in the cell to generate a full expression cassette, from which a full-length protein (e.g., therapeutic protein) is expressed.

The term “therapeutic drug product” refers to any drug that is administered to a patient and effectuates a clinically meaningful response for a given indication. Such drug products are biological, and include, but are not limited to, antibodies, modified cells, cell or gene therapies, viruses, bacteria, nanoparticles, nucleic acids, and biological molecules.

The term “isolated,” when used as a modifier of a composition, means that the composition is provided is a different environment then occurs in nature. For example, the composition can be produced or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

The term “isolated” does not exclude further combinations, for example, an isolated rAAV sequence, or isolated rAAV particle encapsidates an rAAV sequence can be provided in a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells providing a different environment than that occurring in nature.

The term “substantially pure” refers to a preparation comprising at least 50-60% by weight the compound or molecule of interest (e.g., nucleic acid, oligonucleotide, protein, capsid, rAAV particle, etc.). Purity is measured by methods appropriate for the particular compound or molecule of interest (e.g., chromatographic methods, agarose or polyacrylamide gel electrophoresis, HPLC (high-performance liquid chromatography) analysis, and the like). In some embodiments, an rAAV preparation is substantially pure of full rAAV particles when the full particles comprise at least 70% by weight of the preparation.

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

Nucleic acid molecules, expression vectors (e.g., AAV vector genomes), plasmids, including nucleic acid encoding modified or variant AAV capsids of the invention and heterologous nucleic acids may be prepared by using recombinant DNA technology methods. The availability of nucleotide sequence information enables preparation of nucleic acid molecules of the invention by a variety of means. For example, nucleic acid sequences can be made using various standard cloning, recombinant DNA technology, via cell expression and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Ionic strength is a measure of the effective salt composition and electrostatic screening of a solution. Ionic strength effects on a formulation can include electrostatic interactions in a solution and influencing colloidal stability. Ionic strength is typically estimated as shown in Equation 1, in which Cᵢ is the concentration of each ionic species, and Zᵢ is the charge of that species. In certain embodiments a ‘low’ ionic strength is between approximately 25-50 mM, and ‘moderate’ ionic strength between 50-95 mM.

$$IS = \sum {C}_{i} {z}_{i}^{2} / 2 \qquad Eqn . 1$$

### I. Viral Vectors

A viral vector comprising viral nucleic acid encapsidated in a protein capsid can deliver the viral vector nucleic acid to cells or tissues. Depending on the particular vector, the viral vector may further comprise a viral envelope. Examples of viral vectors that can be used for gene delivery include adenovirus vectors, rAAV, retrovirus vectors and herpes simplex vectors.

Different serotypes exist within different types of viruses. The different serotypes can provide for different activities, such as cell or tissue tropism and likelihood of generating a host immune response. The term “serotype” broadly refers to both serologically distinct viruses as well as viruses not serologically distinct that can be within a subgroup or a variant of a given serotype. Serologic distinctiveness can be determined based on the lack of cross-reactivity between antibodies to one capsid as compared to another capsid. Such cross-reactivity differences are usually due to differences in capsid protein sequences/antigenic determinants (e.g., due to VP1, VP2, and/or VP3 sequence differences of AAV serotypes).

As more naturally occurring virus isolates are discovered or capsid mutants generated, there may or may not be serological differences with any of the currently existing serotypes. Thus, in cases where the new virus has no serological difference, this new virus would be a subgroup or variant of the corresponding serotype.

### I.A. Adenovirus Vectors

Adenoviruses are non-enveloped double-stranded DNA viruses. Recombinant adenovirus vectors comprise recombinant adenovirus nucleic acid lacking one or more protein involved in viral replication, and further comprise an adenoviral capsid. Recombinant adenovirus vectors can be produced containing different amounts of adenoviral DNA. The adenovirus (Ad) genome is flanked by hairpin-like inverted terminal repeats (ITRs) varying in length from 30-371 bp at its termini. The ITRs serve as self-priming structures that promote primase-independent DNA replication. A packaging signal located at the left arm of the genome is required for viral genome packaging. (Liu and Seol (2020) BMB Reports; 53 (11): 565-575; and Bulcha et al., (2021) Sig. Transduct. Target Ther. 6:53.)

In certain embodiments, the recombinant adenovirus vector is a third-generation vector, which are also referred to as “gutless” or “helper-dependent”. Gutless vectors can be produced from recombinant adenovirus nucleic acid where all, or substantially all viral sequences, except for the ITRs and the packaging signal, are not present. Gutless adenovirus vectors are high capacity vectors able to accommodate up to about 36 kb of DNA insert. Preferred recombinant adenovirus nucleic acid is about 27 kb to about 37 kb. Stuffer sequences can be added to recombinant adenovirus nucleic acid to increase nucleic acid size and capsid incorporation. Preferred stuffer sequences avoid coding sequences, repetitive sequences, recombination sequences, and immunogenic sequences. (Liu and Seol (2020) BMB Reports, 53 (11): 565-575; Bulcha et al., (2021) Sig. Transduct. Target Ther. 6:53; and Sandig et al., PNAS (2000) 97 (3): 1002-1007, each of which are hereby incorporated by reference herein in their entirety.)

In certain embodiments, recombinant adenovirus vectors can be produced based on rare human serotypes or chimpanzee serotypes. The use of chimpanzee and rare human serotypes may be helpful in reducing host immune response against recombinant adenovirus vectors due to preexisting immunity. (Guo et al., (2018) Human vaccines & immunotherapeutics, 14 (7): 1679-1685 and Bulcha et al., (2021) Sig. Transduct. Target Ther. 6:53.)

Adenovirus vectors can be produced by supplying viral proteins needed for vector production in trans using, for example, appropriate helper viruses or plasmids and cell lines. (Liu and Seol (2020) BMB Reports; 53 (11): 565-575; and Bulcha et al., (2021) Sig. Transduct. Target Ther. 6:53.)

### I.B. Recombinant AAV Vectors

Recombinant adeno-associated viral vector are based on the adeno-associated virus. The adeno-associated virus is a single-strand DNA virus containing a 4.7-kb genome flanked by 145-nt ITRs on both ends of the genome. ITR activity is important for self-priming and packaging, and may also provide additional activity such as promoter activity. AAV 5′ and 3 ITRs can vary in size and the 5′ and 3′ inverted repeats need not be exact inverted repeats.

A rAAV vector contains AAV recombinant nucleic acid and a viral capsid. The rAAV recombinant nucleic acid lacks one or more AAV proteins involved in viral replication. In certain embodiments, the rAAV vector contains an AAV 5′ and/or 3′ ITR along with a DNA insert. In certain embodiments, rAAV nucleic acid comprise a 5′ ITR and/or 3′ ITR independently selected from 5′ and 3′ ITRs provided in AAV1, AAV2, AAV3, AAV4, AAV5, AAV6, AAV7, AAV8, AAV9, AAV10, AAV11, AAV12, AAVrh. 10, AAVrh.74 and AAV3B ITRs. In further embodiments 5′ and 3′ ITRs are present, and both ITRs are from the same serotype genome.

Recombinant adeno-associated viral vectors typically accept inserts of DNA having a size range generally about 4 kb to about 5.2 kb. If needed, stuffer sequences can be used to increase rAAV nucleic acid size and packaging efficiency. In certain embodiments the rAAV nucleic acid including stuffer is less than 5.5 kb. In further embodiments the rAAV nucleic acid including stuffer is less than 5.2 kb, less than 5.1 kb, less than 5.0 kb, less than 4.9 kb, less than 4.8 kb, less than 4.7 kb, less than 4.6 kb; between 4 kb to 5.2 kb, 3.0 kb to 5.5 kb, 4.0 kb to 5.0 kb, or 4.3 kb to 4.8 kb; or about 4.2 kb, about 4.3 kb, about 4.4 kb, about 4.5 kb, about 4.6 kb, about 4.7 kb, about 4.8 kb, about 4.9 kb, or about 5.0 kb. Preferred stuffer sequences avoid coding sequences, repetitive sequences, recombination sequences, and immunogenic sequences.

In certain embodiments the rAAV is a self-complementary adeno-associated virus vector (scAAV) or short hairpin adeno-associated virus vector (shAAV). scAAV and shAAV provide for a double-stranded rAAV nucleic acid that can be incorporated into an AAV caspid. scAAV and shAAV comprise inverted dimeric repeats providing intramolecular double-stranded DNA. scAAV can be produced by mutating an ITR terminal resolution site so that rep fails to nick the terminal resolution site. shAAV can utilize a short hairpin to produce double-stranded AAV nucleic acid. scAAV and shAAV being double-stranded DNA provide an advantage in circumventing the DNA synthesis step required for single-stranded rAAV nucleic acid upon entry into a cell. A potential disadvantage of scAAV and shAAV is the size of DNA inserts that can be incorporated is reduced by about half compared to single-stranded rAAV nucleic acid. (U.S. Pat. No. 10,457,940; Xie et al., Mol Ther. (2017) 25 (6): 1363-1374; and McCarty Mol. Ther. (2008) 16 (10): 1648-1656; each of which are hereby incorporated by reference herein in their entirety.)

Naturally occurring AAV capsids contain viral proteins VP1, VP2 and VP3 in a ratio of about 1:1:10. AAV vectors can be produced where all three viral proteins are based upon a particular serotypes or where one, two or all three viral protein are based on different serotypes.

Recombinant AAV capsid and nucleic acid can be based on the same serotype (or subgroup or variant), or can be different from each other. In certain embodiments, a rAAV nucleic acid has the same serotype genome (e.g., ITRs) as the encapsidating capsid protein.

In different embodiments, the rAAV capsid comprises a protein having a sequence identity of at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.9% or 100% identical to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-218, AAVrh.10, AAVrh.8, AAVHSC, AAV-B1, AAV-AS, or AAV1/rh.10; or VP1 of SEQ ID NO: 1. Recombinant AAV capsids comprising VP1 of SEQ ID NO: 1 is described, for example, in U.S. Pat. No. 9,840,719, and is incorporated herein by reference.

In certain embodiments, AAV capsids comprises VP1, VP2 and VP3 each independently having a sequence identity of at least 80%, at least 90%, at least 95% or 100% to a VP1, VP2 or VP3 of any of AAV1, AAV2, AAV3, AAV4, AAV5, AAV6, AAV7, AAV8, AAV9, AAV10, AAV11, AAV12, AAVrh.74, AAV3B, AAV-218, AAVrh.10, AAVrh.8, AAVHSC, AAV-B1, AAV-AS, AAV1/rh.10, as well as variants (e.g., capsid variants, such as amino acid insertions, additions, substitutions and deletions) thereof. (See, for example, U.S. Pat. Nos. 9,909,142 and 9,840,719 disclosing RHM4-1, RHM15-1, RHM15-2, RHM15-3/RHM15-5, RHM15-4 and RHM15-6; and U.S. Patent Publication No. 2013/0059732.

In certain embodiments, the rAAV capsid comprises VP1 comprising the nucleotide sequence of SEQ ID NO: 1. In a further embodiment the AAV capsid further comprises VP2 comprising the nucleotide sequence of SEQ ID NO: 2. In preferred embodiment, the AAV capsid comprises VP1 comprising the sequence of SEQ ID NO: 1, VP2 comprising the sequence of SEQ ID NO: 2 and VP3 comprising the sequence of SEQ ID NO: 3.

The AAV genome contains two main genes: rep and cap. Transcription from the rep gene is initiated from two different promoters resulting in the production of nonstructural proteins designated Rep78, Rep68, Rep52, and Rep40. The rep proteins function in genome replication and/or encapsidation. The cap gene encodes for structural proteins making up the capsid (VP1, VP2 and Vp3); a non-structural assembly-activating protein (APP), which performs functions related to capsid assembly; and the membrane-associated accessory protein, which may be associated with production phases of the replication cycle. (Maurer and Weitzman (2020) Hum. Gene Ther. 31 (9-10): 499-511, hereby incorporated by reference herein in its entirety.)

AAV requires helper virus functions to complete it replication cycle. Helper virus functions can be supplied by different viruses in permissive cell lines. Permissive cell lines are cell lines able to support viral replication. Examples of helper viruses for AAV include adenovirus, HSV-1, HPV-16, and HBoV1 which can be used in conjunction with, for example, permissive primate cells; and baculovirus which can be used in conjunction with, for example, permissive insect cells such as sf9. (Maurer and Weitzman (2020) Hum. Gene Ther. (2020) 31 (9-10): 499-511 and Meier et al., (2020) Viruses 19; 12 (6): 662, both of which are herein incorporated by reference herein in their entirety.)

Recombinant AAV can be produced by supplying viral proteins needed for vector production in trans using, for example, appropriate helper viruses or plasmids and cell lines. In certain embodiments, rAAV is produced using a rAAV vector genome plasmid. The plasmid comprises that portion of the rAAV nucleic acid ultimately packaged or encapsidated to form a viral (e.g., rAAV) vector. The “plasmid backbone,” contains elements important for propagation and recombinant virus production. Except for possible 3′ ITR and/or 5′ ITR cloning remnants the plasmid backbone is not itself packaged or encapsidated into virus particles.

The vector genome plasmid may contain regions such an origin of replication and a selectable marker. Additional sites that may be present include cloning sites.

### II. Formulations

In certain embodiments, the present disclosure is directed to compositions and methods for the formulation and use of gene therapy products. In certain embodiments, the application relates to rAAV formulations that maintain predictable (stable) freeze-thaw and lyophilization (freeze-drying) performance, and enable longer term shelf storage at temperatures above −60° C. with retention of genome recovery and critical quality attributes. In certain embodiments, the selection of specific components of the rAAV formulation can impact specific critical quality attributes. For example, but not by way of limitation, the inventors show that pH of about 4.5 to about 8 in formulations can be advantageous for enhancing stability, and minimizing degradation at varying storage temperatures (e.g., 2-8° C., −20° C.) and under stress conditions. Moreover, the formulations described herein employ lower ionic strength than in conventional rAAV formulations and enable advantageous storage at higher temperatures without compromising the quality, stability and functionality contrary to generally accepted beliefs (see, Wright, J. Frasier, et al., “Identification of factors that contribute to recombinant AAV2 particle aggregation and methods to prevent its occurrence during vector purification and formulation”, Molecular Therapy, (2005) 12:1, Rodrigues, Gerard A., et al., “Pharmaceutical development of AAV-based gene therapy products for the eye”, Pharmaceutical Research, (2019) 36:29, and Srivastava, Arvind, et al. “Manufacturing challenges and rational formulation development for AAV viral vectors”, Journal of Pharmaceutical Sciences 110.7 (2021): 2609-2624).

In certain embodiments, he inventors disclose low ionic strength (20-50 mM) and moderate ionic strength (50-95 mM) formulations that maintain key critical quality attributes. These attributes are known to correlate to safety and efficacy, and extended shelf life (>1.5 years). An extended shelf life for an rAAV formulation with a CQA profile as shown here is needed in the field given challenges for supply and distribution across the world in liquid and frozen states.

In some embodiments, the formulation disclosed is amenable to lyophilization further extending shelf life and ease of supply and distribution. Contrary to conventional rAAV formulations stored at −80° C., the low ionic strength, sugar containing and low pH (pH 5.2) formulation (F1) exhibits superior stability and quality retention at temperature conditions such as −20° C., −40° C. and −80° C. over an extended storage time period (i.e., from 6 months to 3 years). The moderate ionic strength formulation (F3) exhibits adequate stability and quality retention at temperature conditions such as −20° C., −40° C. and −80° C. over an extended storage time period (i.e., from 6 months to 3 years).

In certain embodiments the ratio of the empty particles in the preparation to the full rAAV particles, also referred to as the E/F ratio, is preferably no more than 1:9 (empty: full), including no more than 1:10, 1:15, 1:20, 1:25, 1:30, 1:35, 1:40, 1:45, 1:50, or any ratio in between, more preferably no less than 1:49. In other embodiments, the ratio of the empty particles in the preparation to the full rAAV particles is preferably no more than 3:7, including no more than 3:8, 3:9, 3:10, 3:11, 3:12, or any ratio in between. In other embodiments, the ratio of the empty particles in the preparation to the full rAAV particles is preferably no more than 0:1.

In certain embodiments, the rAAV formulations of the present disclosure comprise: a plurality of rAAV genomes; a salt or a mixture of salts to adjust the ionic strength of the formulation; a buffering agent; a sugar; and a surfactant. For example, in certain embodiments, the plurality of rAAV genomes are present at a concentration of about 1×10⁹ to about 1×10¹⁵ vg/mL (viral genomes/mL) including 1×10⁹, 1×10¹⁰, 1×10¹¹, 1×10¹², 1×10¹³, 1×10¹⁴, 1×10¹⁵ vg/mL or any concentration in between.

In certain embodiments, the buffering agent in the rAAV formulations of the present disclosure is a non-phosphate buffering agent. In certain embodiments, the non-phosphate buffering agent is sodium acetate. In certain embodiments, the non-phosphate buffering agent is Tris (Tris(hydroxymethyl)aminomethane), HEPES, sodium citrate, succinate, and mixtures, thereof.

In certain embodiments, the non-phosphate buffering agent is present at a concentration of about 1 mM to about 50 mM (for low ionic strength) such as 1 mM, 5 mM, 10 mM, 15 mM, 20 mM, 25 mM, 30 mM, 35 mM, 40 mM, 45 mM, 50 mM, or any concentration in between. In certain embodiments, the non-phosphate buffering agent is present at a concentration of about 50 mM to about 100 mM (for moderate ionic strength) including 50 mM, 55 mM, 60 mM, 65 mM, 70 mM, 75 mM, 80 mM, 85 mM, 90 mM, 95 mM, 100 mM, or any concentration in between. In certain embodiments, the non-phosphate buffering agent is sodium acetate present at a concentration of about 25 mM.

In certain embodiments, the salt determining the ionic strength of the formulations of the present disclosure, is sodium chloride. In certain embodiments, the sodium chloride is present at a concentration of about 1 mM to about 180 mM including 1 mM, 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM or any concentration in between. In certain embodiments, the sodium chloride is preferably at a concentration of about 60 mM. In certain embodiments, the sodium chloride is present most preferably at a concentration of about 25 mM.

In certain embodiments, the salt used as an excipient in the formulations of the present disclosure, is magnesium chloride. In certain embodiments, the magnesium chloride is present at a concentration of about 1 mM to about 10 mM including 1 mM, 2 mM, 3 mM, 4 mM, 5 mM, 6 mM, 7 mM, 8 mM, 9 mM, 10 mM, or any concentration in between. In certain embodiments, the magnesium chloride is present at a concentration of about 5 mM.

In certain embodiments, the rAAV formulations of the present application comprise a sugar. In certain embodiments, the sugar content of the formulation is about 0% to about 20% including 0%, 1%, 2%, 3%, 4%, 5%, 6%, 7%, 8%, 9%, 10%, 11%, 12%, 13%, 14%, 15%, 16%, 17%, 18%, 19%, 20%, or any concentration in between. In certain embodiments, the sugar content of the formulation is preferably about 5.4%. In certain embodiments, the sugar is sucrose. In certain embodiments, the sugar content, including the sucrose content, of the formulation is about 3%.

In certain embodiments, the rAAV formulations of the present disclosure comprise a surfactant. In certain embodiments, the surfactant is present at a concentration of between about 0 and 0.05% including 0%, 0.01%, 0.02%, 0.03%, 0.04%, 0.05%, or any concentration in between. In certain embodiments, the surfactant is Poloxamer 188 (Pluronic® f-68 or P188). In certain embodiments, the surfactant(s) is selected from Polysorbate 20 (PS20), Polysorbate 80 (PS80) or a combination, thereof. In certain embodiments, the Poloxamer 188 is present most preferably at a concentration of about 0.01%. In certain embodiments, the Poloxamer 188 is present at a concentration of about 0.001%.

In certain embodiments, the rAAV formulations of the present disclosure contains a stabilizer. In certain embodiments, the stabilizer is an amino acid. In certain embodiments, the amino acid is arginine or histidine.

In certain embodiments, the rAAV formulations of the present disclosure are prepared such that the pH is from about 4.5 to about 8 including 4.5, 5, 5.5, 6, 6.5, 7, 7.5, 8, or any pH in between. In certain embodiments, the pH is most preferably about 5.2. In certain embodiments, the pH is about 7.4.

In certain embodiments, the rAAV formulations of the present disclosure comprise about 25 mM Na Acetate, about 25 mM NaCl, about 5 mM Mg2Cl, pH of about 5.2, about 5.4% Sucrose, and about 0.01% Poloxamer 188, referred herein as “Formulation 1” or “F1”.

In certain embodiments, the buffering agent in the rAAV formulations of the present disclosure is a phosphate buffering agent. In certain embodiments, the phosphate buffering agent is sodium phosphate. In certain embodiments, phosphate buffering agent is present at a concentration of about 1 mM to about 50 mM including 1 mM, 10 mM, 20 mM, 30 mM, 40 mM, 50 mM, or any concentration in between. In certain embodiments, the phosphate buffering agent is sodium phosphate present at about 10 mM.

In certain embodiments, the rAAV formulations of the present disclosure comprise about 10 mM sodium phosphate, 60 mM sodium chloride, pH of about 7.4, 3% sucrose, 0.001% Poloxamer 188, referred herein as “Formulation 3” or “F3”.

In certain embodiments, the formulation maintains its pH within +/−0.1 pH when stored at −40° C. for 6 months. In certain embodiments, the formulation maintains its pH within +/−0.1 pH when stored at −40° C. for 2 months. In certain embodiments, the formulation maintains its pH within +/−0.1 pH when stored at −20° C. for 6 months. In certain embodiments, the formulation maintains its pH within +/−0.1 pH when stored at −20° C. for 2 months. In certain embodiments, the formulation maintains its pH within +/−0.1 pH when stored at 5° C. for 6 months. In certain embodiments, the formulation maintains its pH within +/−0.1 pH when stored at 5° C. for 2 months.

In certain embodiments of the present disclosure, the rAAV formulation exhibits a glass transition temperature of freeze concentrate of about −39° C. In certain embodiments, it is beneficial to store biologics below glass transition for enhanced stability with minimized mobility. Increased Tg′ (glass transition temperature) also enables storage at temperatures higher than −80° C. which is favorable and highly desirable considering the ease of supply chain practices including handling/shipping and storage at higher temperatures. It is also amenable as an adoptable approach since it maximizes the sterility assurance (i.e., low temperature storage can compromise container closure systems, as −80° C. is generally below the glass transition of plastics and rubber due to which plastic generally becomes brittle at these low temperatures).

In certain embodiments, the rAAV formulations of the present disclosure are prepared such that the formulation exhibits similar or better recovery of vector genomes (genome recovery) after storage at different conditions compared to that of the genome recovery same formulation prior to storage. In certain embodiments, the rAAV formulations of the present disclosure are prepared such that the formulation exhibits a genome recovery of about 50% to about 150% (including 50%, 60%, 70%, 80%, 90%, 100%, 110%, 120%, 130%, 140%, 150% or any percent in between) after storage at 5° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the genome recovery of the same formulation prior to storage. Genome recovery can be measured as described in the examples below.

In certain embodiments, the formulation exhibits a genome recovery of about 50% to about 150% (including 50%, 60%, 70%, 80%, 90%, 100%, 110%, 120%, 130%, 140%, or 150% or any percent in between) after storage at −40° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the genome recovery of the same formulation prior to storage.

In certain embodiments, the formulation exhibits a genome recovery of about 50% to about 150% (including 50%, 60%, 70%, 80%, 90%, 100%, 110%, 120%, 130%, 140%, or 150% or any percent in between) after storage at −20° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the genome recovery of the same formulation prior to storage.

In certain embodiments, the formulation exhibits a genome recovery of about 50% to about 150% (including 50%, 60%, 70%, 80%, 90%, 100%, 110%, 120%, 130%, 140%, or 150% or any percent in between) after storage at 5° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the genome recovery of the same formulation prior to storage.

In certain embodiments, the rAAV formulations of the present disclosure are prepared such that the formulation retains similar or better potency after storage at different conditions relative to that of the same formulation prior to storage (relative potency). In certain embodiments, the rAAV formulations of the present disclosure are prepared such that the formulation exhibits a relative potency of about 50% to about 150% (including 50%, 60%, 70%, 80%, 90%, 100%, 110%, 120%, 130%, 140%, 150% or any percent in between) after storage at 5° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the relative potency of the same formulation prior to storage.

In certain embodiments, the formulation exhibits a relative potency of about 50% to about 150% (including 50%, 60%, 70%, 80%, 90%, 100%, 110%, 120%, 130%, 140%, 150% or any percent in between) after storage at −20° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the relative potency of the same formulation prior to storage.

In certain embodiments, the formulation exhibits a relative potency of about 50% to about 150% (including 50%, 60%, 70%, 80%, 90%, 100%, 110%, 120%, 130%, 140%, or 150% or any percent in between) after storage at −40° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the relative potency of the same formulation prior to storage.

In certain embodiments, the formulation exhibits a relative potency of about 50% to about 150% (including 50%, 60%, 70%, 80%, 90%, 100%, 110%, 120%, 130%, 140%, or 150% or any percent in between) after storage at −80° C. for about 1 day, 3 days, 5 days, 1 week, 2 weeks, 3 weeks, 4 weeks, 1 month, 2 months, 3 months, 4 months, 5 months, 6 months, 7 months, 8 months, 9 months, 10 months, 11 months, 1 year, 2 years or 3 years compared to the relative potency of the same formulation prior to storage.

In certain embodiments, the rAAV formulations of the present disclosure are prepared such that the formulation is amenable to intravenous, intraarterial, intraocular, intrathecal, or intracerebral injection. In certain embodiments, the formulation is a liquid. In certain embodiments, the formulation is amenable to lyophilization. In certain embodiments, the formulation is in a unit dosage container. In certain embodiments, the unit dosage container is a vial. In certain embodiments, the vial is a sealed glass vial. In certain embodiments, the vial is a cyclic olefin polymer vial, also known as a CZ vial.

In certain embodiments, the rAAV formulations of the present disclosure include various viral strains or serotypes. In certain, non-limiting, embodiments, an rAAV can be based upon any AAV genome, including, but not limited to AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1, DJ, DJ8, NP59, Anc-80 and variants thereof, including the variants of AAV capsids set forth in Pulicherla et al., Mol. Ther., 19 (6) 1070-1078 (2011) (describing AAV9 variants including AAV9.47 among others), U.S. Pat. No. 7,906,111 (describing AAV9 (hu14) among others), U.S. Pat. No. 10,532,111 (describing NP59 among others), U.S. Pat. No. 10,738,087 (describing Anc80 among others), WO2012/145601, WO2013/158879, WO2015/013313, WO2018/156654, U.S. Pat. No. 9,840,719 (describing RHM4-1), U.S. Pat. Nos. 7,749,492, 7,588,772 (describing DJ and DJ8), and U.S. Pat. No. 9,587,282, all of which are incorporated herein by reference in their entireties. rAAV vectors therefore include gene/protein sequences identical to gene/protein sequences characteristic for a particular serotype, as well as mixed serotypes.

In certain embodiments, the formulation pH is adjusted prior to administration.

### III. rAAV Comprising a Gene of Interest (GOI)

In certain embodiments, the rAAV comprises a gene of interest (GOI). In certain non-limiting embodiments, the GOI comprises a nucleic acid sequence encoding a therapeutic protein or an inhibitory nucleic acid sequence. In certain embodiments, the GOI can be introduced/transferred by way of vector, including rAAV transduction or transfection into a cell. In certain embodiments, the introduced GOI can also exist in the recipient cell or host organism extrachromosomally, or only transiently.

In certain embodiments, the GOI encodes a protein (e.g., therapeutic protein) that is provided in modified or truncated forms or the GOI is provided in multiple constructs, delivered by separate and multiple AAV vectors.

In certain embodiments, the GOI is provided as a truncated variant that maintains functionality of the encoded protein (e.g., therapeutic protein), including removal of portions unnecessary for function, such that the GOI is reduced in size for packaging in an AAV vector.

In certain embodiments, the GOI is provided in split AAV vectors, each providing nucleic acid encoding different portions of a protein (e.g., therapeutic protein), thus delivering multiple portions of a protein (e.g., therapeutic protein) which assemble and function in the mammalian rAAV producing cell of the present disclosure.

In certain embodiments, the GOI is provided by dual AAV vectors using overlapping, trans-splicing or hybrid trans-splicing dual vector technology. In certain embodiments, two overlapping AAV vectors are used which combine in the mammalian rAAV producing cell of the present disclosure to generate a full expression cassette, from which a full-length protein (e.g., therapeutic protein) is expressed.

Non-limiting examples of heterologous nucleic acids encoding gene products (e.g., therapeutic proteins) which are useful in accordance with the invention include those that may be used in the treatment of a disease or disorder including, but not limited to, “hemostasis” or blood clotting disorders including hemophilia A, hemophilia A patients with inhibitory antibodies, hemophilia B, deficiencies in coagulation Factors, VII, VIII, IX and X, XI, V, XII, II, von Willebrand factor, combined FV/FVIII deficiency, thalassemia, vitamin K epoxide reductase CI deficiency, gamma-carboxylase deficiency; anemia, bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e., FXa inhibitors); and platelet disorders including, Bernard Soulier syndrome (BSS), Glanzman thromblastemia (GT), and storage pool deficiency.

In certain embodiments, the disease or disorder affects or originates in the central nervous system (CNS). In certain embodiments, the disease is a neurodegenerative disease. In certain embodiments, the CNS or neurodegenerative disease is Alzheimer's Disease (AD), Huntington's Disease (HD), amyotrophic lateral sclerosis (ALS), hereditary spastic hemiplegia, primary lateral sclerosis (PLS), spinal muscular atrophy (SMA), Kennedy's disease (Spinal and Bulbar Muscular Atrophy or SBMA), a polyglutamine repeat disease, or Parkinson's disease. In certain embodiments, the CNS or neurodegenerative disease is a polyglutamine repeat disease. In certain embodiments, the polyglutamine repeat disease is a spinocerebellar ataxia (SCA1, SCA2, SCA3, SCA6, SCA7, or SCA17).

In certain embodiments, the rAAV particles present in the formulations of the instant disclosure comprise a heterologous nucleic acid encoding a gene product selected from the group consisting of insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), TGFp, activins, inhibins, bone morphogenic protein (BMP), nerve growth factor (NGF), brain-derived neurotrophic factor (BDNF), neurotrophins NT-3 and NT4/5, ciliary neurotrophic factor (CNTF), glial cell line derived neurotrophic factor (GDNF), neurturin, agrin, netrin-1 and netrin-2, hepatocyte growth factor (HGF), ephrins, noggin, sonic hedgehog and tyrosine hydroxylase.

In certain embodiments, the rAAV particles present in the formulations of the instant disclosure comprise a heterologous nucleic acid encoding a gene product selected from the group consisting of thrombopoietin (TPO), interleukins (IL1 through IL-17), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand, IgG, IgM, IgA, IgD and IgE, chimeric immunoglobulins, humanized antibodies, single chain antibodies, T cell receptors, chimeric T cell receptors, single chain T cell receptors, class I and class II MHC molecules.

In certain embodiments, the rAAV particles present in the formulations of the instant disclosure comprise a heterologous nucleic acid encoding a gene product selected from the group consisting of carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-6-phosphatase, porphobilinogen deaminase, factor V, factor VIII, factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase (GLDC), RPE65, H-protein, T-protein, a cystic fibrosis transmembrane regulator (CFTR) sequence, and a dystrophin cDNA sequence

In certain embodiments, the rAAV particles present in the formulations of the instant disclosure comprise a heterologous nucleic acid encoding a polypeptide, a nucleic acid that encodes a protein or is transcribed into a transcript of interest, or nucleic acid, selected from the group consisting of a siRNA, an antisense molecule, miRNA, a ribozyme and a shRNA.

In certain embodiments, the rAAV particles present in the formulations of the instant disclosure comprise a heterologous nucleic that encodes a protein selected from the group consisting of GAA (acid alpha-glucosidase) for treatment of Pompe disease; ATP7B (copper transporting ATPase2) for treatment of Wilson's disease; alpha galactosidase for treatment of Fabry's disease; ASS1 (arginosuccinate synthase) for treatment of citrullinemia Type 1; beta-glucocerebrosidase for treatment of Gaucher disease Type 1; beta-hexosaminidase A for treatment of Tay Sachs disease; SERPING1 (C1 protease inhibitor or C1 esterase inhibitor) for treatment of hereditary angioedema (HAE), also known as C1 inhibitor deficiency type I and type II); and glucose-6-phosphatase for treatment of glycogen storage disease type I (GSDI).

In certain embodiments, a heterologous nucleic acid present in the rAAV present in the formulations of the instant disclosure encodes CFTR (cystic fibrosis transmembrane regulator protein), a blood coagulation (clotting) factor (Factor XIII, Factor IX, Factor VIII, Factor X, Factor VII, Factor VIIa, protein C, etc.) a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1 or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, and variants thereof.

Nucleic acid molecules, vectors including cloning, expression vectors (e.g., vector genomes) and plasmids, may be prepared using recombinant DNA technology methods. The availability of nucleotide sequence information enables preparation of nucleic acid molecules by a variety of means. For example, a heterologous nucleic acid encoding Factor IX (FIX) comprising a vector or plasmid can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

### IV. Sequences

SEQ ID NO: 1
MAADGYLPDWLEDNLSEGIREWWDLKPGAPKPKANQQKQDNGRGLVLPGYKYLGPFNGLDKGEPVNAADAAAL
EHDKAYDQQLQAGDNPYLRYNHADAEFQERLQEDTSFGGNLGRAVFQAKKRVLEPLGLVESPVKTAPGKKRPV
EPSPQRSPDSSTGIGKKGQQPAKKRLNFGQTGDSESVPDPQPIGEPPAAPSGVGPNTMAAGGGAPMADNNEGA
DGVGSSSGNWHCDSTWLGDRVITTSTRTWALPTYNNHLYKQISNGTSGGSTNDNTYFGYSTPWGYFDFNRFHC
HFSPRDWQRLINNNWGFRPKRLNFKLFNIQVKEVTQNEGTKTIANNLTSTIQVFTDSEYQLPYVLGSAHQGCL
PPFPADVFMIPQYGYLTLNNGSQAVGRSSFYCLEYFPSQMLRTGNNFEFSYNFEDVPFHSSYAHSQSLDRLMN
PLIDQYLYYLSRTQSTGGTAGTQQLLFSQAGPNNMSAQAKNWLPGPCYRQQRVSTTLSQNNNSNFAWTGATKY
HLNGRDSLVNPGVAMATHKDDEERFFPSSGVLMFGKQGAGKDNVDYSSVMLTSEEEIKTTNPVATEQYGVVAD
NLQQQNAAPIVGAVNSQGALPGMVWQNRDVYLQGPIWAKIPHTDGNFHPSPLMGGFGLKHPPPQILIKNTPVP
ADPPTTFNQAKLASFITQYSTGQVSVEIEWELQKENSKRWNPEIQYTSNYYKSTNVDFAVNTEGTYSEPRPIG
TRYLTRNL
SEQ ID NO: 2
TAPGKKRPVEPSPQRSPDSSTGIGKKGQQPAKKRLNFGQTGDSESVPDPQPIGEPPAGPSGLGSGTMAAGGGA
PMADNNEGADGVGSSSGNWHCDSTWLGDRVITTSTRTWALPTYNNHLYKQISNGTSGGSTNDNTYFGYSTPWG
YFDFNRFHCHFSPRDWQRLINNNWGFRPKRLNFKLFNIQVKEVTQNEGTKTIANNLTSTIQVFTDSEYQLPYV
LGSAHQGCLPPFPADVFMIPQYGYLTLNNGSQAVGRSSFYCLEYFPSQMLRTGNNFEFSYNFEDVPFHSSYAH
SQSLDRLMNPLIDQYLYYLSRTQSTGGTAGTQQLLFSQAGPNNMSAQAKNWLPGPCYRQQRVSTTLSQNNNSN
FAWTGATKYHLNGRDSLVNPGVAMATHKDDEERFFPSSGVLMFGKQGAGKDNVDYSSVMLTSEEEIKTTNPVA
TEQYGVVADNLQQQNAAPIVGAVNSQGALPGMVWQNRDVYLQGPIWAKIPHTDGNFHPSPLMGGFGLKHPPPQ
ILIKNTPVPADPPTTFNQAKLASFITQYSTGQVSVEIEWELQKENSKRWNPEIQYTSNYYKSTNVDFAVNTEG
TYSEPRPIGTRYLTRNL
SEQ ID NO: 3
MAAGGGAPMADNNEGADGVGSSSGNWHCDSTWLGDRVITTSTRTWALPTYNNHLYKQISNGTSGGSTNDNTYF
GYSTPWGYFDFNRFHCHFSPRDWQRLINNNWGFRPKRLNFKLFNIQVKEVTQNEGTKTIANNLTSTIQVFTDS
EYQLPYVLGSAHQGCLPPFPADVFMIPQYGYLTLNNGSQAVGRSSFYCLEYFPSQMLRTGNNFEFSYNFEDVP
FHSSYAHSQSLDRLMNPLIDQYLYYLSRTQSTGGTAGTQQLLFSQAGPNNMSAQAKNWLPGPCYRQQRVSTTL
SQNNNSNFAWTGATKYHLNGRDSLVNPGVAMATHKDDEERFFPSSGVLMFGKQGAGKDNVDYSSVMLTSEEEI
KTTNPVATEQYGVVADNLQQQNAAPIVGAVNSQGALPGMVWQNRDVYLQGPIWAKIPHTDGNFHPSPLMGGFG
LKHPPPQILIKNTPVPADPPTTFNQAKLASFITQYSTGQVSVEIEWELQKENSKRWNPEIQYTSNYYKSTNVD
FAVNTEGTYSEPRPIGTRYLTRNL

### EMBODIMENTS

The application includes, but is not limited to, the following numbered embodiments:

Embodiment 1. A stable formulation comprising a therapeutic drug product and:
(a) 1-100 mM one or more salts;
(b) a buffering agent;
(c) 0.001-0.05% by weight one or more non-ionic surfactants;
(d) 1-10% by weight one or more sugars; and
(e) water,
(f) optionally, one or more amino acids, and
   wherein the composition has a pH of 4.5-7.5 (such as 4.5, 5, 5.5, 6, 6.5, 7, 7.5, or any pH in between), and 60% or more (such as 60%, 70%, 80%, 90%, 100% or any percent in between) of the drug product is recovered after the formulation is frozen and thawed.

Embodiment 2. The stable formulation of Embodiment 1, wherein the therapeutic drug product is a recombinant adeno-associated virus (AAV or rAAV).

Embodiment 3. The stable formulation of any one of Embodiments 1-2, wherein the buffering agent comprises a non-phosphate buffering agent.

Embodiment 4. The stable formulation of Embodiment 3, wherein the non-phosphate buffering agent is sodium acetate.

Embodiment 5. The stable formulation of any one of Embodiments 3-4, wherein the non-phosphate buffering agent is present in the formulation at a concentration of 1 mM to about 50 mM.

Embodiment 6. The stable formulation of any one of Embodiments 1-5, wherein the one or more salts includes 10-100 mM sodium chloride.

Embodiment 7. The stable formulation of Embodiment 6, wherein the one or more salts comprises a first salt and a second salt.

Embodiment 8. The stable formulation of Embodiment 7, wherein the first salt is sodium chloride and the second salt is magnesium chloride.

Embodiment 9. The stable formulation of Embodiment 6, wherein the sodium chloride is present in the formulation at a concentration of about 25 mM.

Embodiment 10. The stable formulation of Embodiment 8, wherein the magnesium chloride is present in the formulation at a concentration of about 1 mM to about 10 mM.

Embodiment 11. The stable formulation of Embodiment 10, wherein the magnesium chloride is present at a concentration of about 5 mM.

Embodiment 12. The stable formulation of any one of Embodiments 1-11, wherein the sugar is sucrose.

Embodiment 13. The stable formulation of Embodiment 12, wherein the sucrose content of the formulation is 5.4%.

Embodiment 14. The stable formulation of any one of Embodiments 1-13, wherein the non-ionic surfactant is present in the formulation at a concentration of about 0.01%.

Embodiment 15. The stable formulation of Embodiment 14, wherein the surfactant is poloxamer 188, polysorbate 20, polysorbate 80, or poloxamer 407.

Embodiment 16. The stable formulation of Embodiment 15, wherein the surfactant is polysorbate selected from the group consisting of polysorbate 20 or polysorbate 80.

Embodiment 17. The stable formulation of Embodiment 1, wherein the pH is about 5.2.

Embodiment 18. The stable formulation of any one of Embodiments 1-2, wherein the buffering agent is a phosphate buffering agent.

Embodiment 19. The stable formulation of Embodiment 18, wherein the phosphate buffering agent is sodium phosphate.

Embodiment 20. The stable formulation of Embodiment 19, wherein sodium phosphate is present at about 10 mM.

Embodiment 21. The stable formulation of any one of Embodiments 1-20, wherein the formulation is lyophilized.

Embodiment 22. The stable formulation of any one of Embodiments 1-20, wherein the formulation is liquid.

Embodiment 23. The stable formulation of any one of Embodiments 1-22, wherein the therapeutic drug product is capable of increasing protein levels in a cell of GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

Embodiment 24. A stable formulation comprising:
(a) a recombinant adeno-associated virus (rAAV);
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5, such as a pH of 5, 5.1, 5.2, 5.3, 5.4, 5.5, or any pH in between.

Embodiment 25. A stable formulation comprising:
(a) a recombinant adeno-associated virus (rAAV);
(b) 10 mM Tris;
(c) 75 mM sodium chloride;
(d) 0.005% by weight poloxamer 188;
(e) 5% by weight sucrose;
(f) water; and
(g) at a pH of 7.3.

Embodiment 26. A stable formulation, wherein the formulation comprises:
(a) a recombinant adeno-associated virus (rAAV);
(b) 10 mM sodium citrate;
(c) 75 mM sodium chloride;
(d) 0.001% by weight poloxamer 188;
(e) 7% by weight sucrose;
(f) water; and
(g) at a pH of 6.0.

Embodiment 27. The stable formulation of Embodiments 24-26, wherein the rAAV comprises a capsid derived from one or more AAVs selected from the group consisting of AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1 (VP1, SEQ ID NO: 1, or VP2, SEQ ID NO: 2, or VP3, SEQ ID NO: 3), DJ, DJ8, NP59, Anc-80, and variants thereof.

Embodiment 28. The stable formulation of Embodiments 24-27, wherein the rAAV comprises a transgene that encodes a polypeptide, or a nucleic acid selected from the group consisting of a siRNA, an antisense molecule, miRNA, a ribozyme and a shRNA.

Embodiment 29. The stable formulation of Embodiment 28, wherein the rAAV comprises the transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

Embodiment 30. The stable formulation of Embodiment 29, wherein the rAAV comprises the transgene that encodes GAA (acid alpha-glucosidase).

Embodiment 31. The stable formulation of Embodiment 29, wherein the rAAV comprises the transgene that encodes ATP7B (copper transporting ATPase2).

Embodiment 32. The stable formulation of Embodiment 29, wherein the rAAV comprises the transgene that encodes alpha galactosidase A (GLA).

Embodiment 33. The stable formulation of Embodiment 29, wherein the rAAV comprises the transgene that encodes ASS1 (arginosuccinate synthase).

Embodiment 34. The stable formulation of Embodiment 29, wherein the rAAV comprises the transgene that encodes beta-glucocerebrosidase.

Embodiment 35. The stable formulation of Embodiment 29, wherein the rAAV comprises the transgene that encodes beta-hexosaminidase A.

Embodiment 36. The stable formulation of Embodiment 29, wherein the AAV comprises the transgene that encodes SERPING1 (C1 protease inhibitor or C1 esterase inhibitor).

Embodiment 37. The stable formulation of Embodiment 29, wherein the AAV comprises the transgene that encodes glucose-6-phosphatase.

Embodiment 38. The stable formulation of Embodiment 29, wherein the AAV comprises the transgene that encodes CFTR (cystic fibrosis transmembrane regulator protein).

Embodiment 39. The stable formulation of Embodiment 29, wherein the AAV comprises the transgene that encodes a blood coagulation (clotting) factor (e.g., Factor XIII, Factor IX, Factor VIII, Factor X, Factor VII, Factor VIIa, protein C).

Embodiment 40. The stable formulation of any of Embodiments 1 to 39, wherein after the composition is frozen for a duration at a temperature above −80° C. and up to −10° C., 60% or more of the therapeutic drug product or rAAV is recovered after thawing compared to the same product stored at −80° C. for same duration.

Embodiment 41. The stable formulation of any of Embodiments 1 to 39, wherein after the composition is frozen for a duration at a temperature above −80° C. and up to −10° C., such as a temperature of −70° C. to −15° C., −60° C. to −20° C., −50° C. to −30° C., or −45° C. to −35° C., the drug product or rAAV exhibits at least 50% relative potency compared to the same product stored at −80° C. for the same duration.

Embodiment 42. The stable formulation of any of Embodiments 1 to 39, wherein after the composition is frozen for a duration at a temperature above −80° C. and up to −10° C., the drug product or rAAV exhibits a glass transition temperature of freeze concentrate of −45° C. to −35° C., such as −45° C., −44° C., −43° C., −42° C., −41° C., −40° C., −39° C., −38° C., −37° C., −36° C. or −35° C.

Embodiment 43. The stable formulation of any one of Embodiments 1-39, wherein the composition is lyophilized.

Embodiment 44. The stable formulation of any one of Embodiments 1-42, wherein the composition is liquid.

Embodiment 45. A lyophilized composition, wherein the composition is substantially dehydrated and is capable of forming the formulation of any one of the preceding Embodiments by reconstituting the lyophilized composition with diluent.

Embodiment 46. The lyophilized composition according to Embodiment 45, wherein the reconstituting comprises combining the lyophilized composition with water for injection (WFI).

Embodiment 47. The stable formulation of any one of Embodiments 1-43 or the lyophilized composition of any one of Embodiments 44-46, wherein the formulation or composition is present in a unit dosage container.

Embodiment 48. The stable formulation or the lyophilized composition of Embodiment 47, wherein the unit dosage container is a vial.

Embodiment 49. The stable formulation or the lyophilized composition of Embodiment 48, wherein the vial is a sealed glass vial.

Embodiment 50. A method of reducing degradation of a therapeutic drug product after freeze-thawing said product, the method comprising preparing a composition comprising:
(a) the therapeutic drug product;
(b) 1-100 mM one or more salts;
(c) a buffering agent;
(d) 0.001-0.05% by weight one or more non-ionic surfactants;
(e) 3-10% by weight one or more sugars; and
(f) water;
(g) at a pH of 4.5 to 7; and
(h) optionally, one or more amino acids, and
   freezing said composition for a duration at a temperature above −80° C. and up to −10° C., such as a temperature of −70° C. to −15° C., −60° C. to −20° C., −50° C. to −30° C., or −45° C. to −35° C., wherein 60% or more of the therapeutic drug product is recovered after thawing compared to the same product stored at −80° C. for same duration.

Embodiment 51. A method of reducing degradation of a therapeutic drug product after freeze-thawing said product, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein 60% or more of the therapeutic drug product is recovered after thawing compared to the same product stored at −80° C. for same duration.

Embodiment 52. A method of retaining at least 50% relative potency of a therapeutic drug product after freeze-thawing said product, the method comprising preparing a composition comprising:
(a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −60° C. and up to −10° C., such as −60° C. to −10° C., −50° C. to −20° C., −45° C. to −25° C., or −30° C. to −40° C., wherein after thawing, said drug product exhibits at least 50% relative potency compared to the same product stored at −80° C. for the same duration.

Embodiment 53. A method of reducing degradation of a therapeutic drug product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose;
(f) water;
(g) at a pH of 7.3; and
   lyophilizing said composition and storing for a duration at a temperature between −20° C. and 8° C., such as a temperature of −20° C. to 0° C. or 0° C. to 8° C., wherein 60% or more of the therapeutic drug product is recovered after reconstituting lyophilized composition compared to the same product stored at −80° C. without lyophilization.

Embodiment 54. A method of reducing degradation of a therapeutic drug product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   lyophilizing said composition and storing for a duration at a temperature between −20° C. and 8° C., such as a temperature of −20° C. to 0° C. or 0° C. to 8° C., wherein 60% or more of the therapeutic drug product is recovered after reconstituting lyophilized composition compared to the same product stored at −80° C. without lyophilization.

Embodiment 55. A method of retaining at least 50% relative potency of a therapeutic drug product, the method comprising preparing a composition comprising:
(a) the therapeutic product;
(b) 10 mM Tris;
(c) 75 mM sodium chloride;
(d) 0.005% by weight poloxamer 188;
(e) 5% by weight sucrose; and
(f) water;
(g) at a pH of 7.3; and
   lyophilizing said composition and storing for a duration at a temperature between −20° C. and 8° C., such as a temperature of −20° C. to 0° C. or 0° C. to 8° C., wherein said drug product exhibits at least 50% relative potency after reconstituting lyophilized composition compared to the same product stored at −80° C. without lyophilization.

Embodiment 56. A method of retaining at least 50% relative potency of a therapeutic drug product, the method comprising preparing a composition comprising:
(a) the therapeutic product;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   lyophilizing said composition and storing for a duration at a temperature between −20° C. and 8° C., such as a temperature of −20° C. to 0° C. or 0° C. to 8° C., wherein said drug product exhibits at least 50% relative potency after reconstituting lyophilized composition compared to the same product stored at −80° C. without lyophilization.

Embodiment 57. The method of any one of Embodiments 50-56, wherein the therapeutic drug product is a recombinant adeno-associated virus (rAAV).

Embodiment 58. A method of reducing deamination of amino acids on capsid of an rAAV, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5.

Embodiment 59. A method of reducing degradation of viral proteins of an rAAV product after freezing and thawing the composition, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5; and
   storing said frozen composition for a duration at a temperature between −80° C. and up to −10° C., such as a temperature of −70° C. to −15° C., −60° C. to −20° C., −50° C. to −30° C., or −45° C. to −35° C., wherein the change in the ratio VP1: VP3 is less than 20% in the thawed composition compared to before freezing said composition.

Embodiment 60. A method of reducing deamination of amino acids on capsid of an rAAV, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3.

Embodiment 61. A method of reducing deamination of amino acids on capsid of an rAAV, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water; and
(g) at a pH of 6.0.

Embodiment 62. A method of reducing subvisible particle (SVP) concentration of an rAAV product after freeze-thawing said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for at least one year at −20° C., wherein after thawing, SVP concentration is less than 100 particles per mL for particles 10 μm or larger, and less than 10 particles per mL for particles 25 μm or larger.

Embodiment 63. A method of reducing subvisible particle (SVP) concentration of an rAAV product after freeze-thawing said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for at least one year at −20° C., wherein after thawing, SVP concentration is less than 50 particles per mL for particles 10 μm or larger, and less than 5 particles per mL for particles 25 μm or larger

Embodiment 64. A method of reducing subvisible particle (SVP) concentration of an rAAV product after 5 freeze-thaw cycles of said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein said composition is thawed and refrozen five times, and upon the fifth thaw, SVP concentration is less than 100 particles per mL for particles 10 μm or larger, and less than 10 particles per mL for particles 25 μm or larger.

Embodiment 65. A method of reducing subvisible particle (SVP) concentration of an rAAV product after 5 freeze-thaw cycles of said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein said composition is thawed and refrozen five times, and upon the fifth thaw, SVP concentration is less than 50 particles per mL for particles 10 μm or larger, and less than 5 particles per mL for particles 25 μm or larger.

Embodiment 66. A method of reducing high molecular weight species (HMW) in an rAAV product after 5 freeze-thaw cycles of said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for a duration at a temperature above −80° C. and up to −10° C., such as a temperature of −70° C. to −15° C., −60° C. to −20° C., −50° C. to −30° C., or −45° C. to −35° C., wherein said composition is thawed and refrozen five times, and upon the fifth thaw, HMW species increase by less than 3% by weight compared to the same product at T0 as measured by analytical ultracentrifugation.

Embodiment 67. A method of reducing high molecular weight species (HMW) in an rAAV product after freezing and thawing said product, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water;
(h) at a pH of 5 to 5.5; and
   freezing said composition for at least one year at −20° C., wherein after thawing, HMW species increase by less than 3% by weight compared to the same product at T0 as measured by analytical ultracentrifugation.

Embodiment 68. A method of reducing degradation of viral proteins of an rAAV product after lyophilizing the product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; and storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to before lyophilization.

Embodiment 69. A method of reducing degradation of viral proteins of an rAAV product after lyophilizing the product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to before lyophilization.

Embodiment 70. A method a method of reducing subvisible particle (SVP) concentration of an rAAV product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 100 particles per mL for particles 10 μm or larger, and less than 10 particles per mL for particles 25 μm or larger.

Embodiment 71. A method a method of reducing subvisible particle (SVP) concentration of an rAAV product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 50 particles per mL for particles 10 μm or larger, and less than 5 particles per mL for particles 25 μm or larger.

Embodiment 72. A method a method of reducing subvisible particle (SVP) concentration of an rAAV product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose;
(f) water;
(g) at a pH of 6.0; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 100 particles per mL for particles 10 μm or larger, and less than 10 particles per mL for particles 25 μm or larger.

Embodiment 73. A method a method of reducing subvisible particle (SVP) concentration of an rAAV product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   storing said lyophilized composition for a duration at a temperature between −20° C. and 8° C., such as a temperature of −20° C. to 0° C. or 0° C. to 8° C., wherein the formulation is subjected to one lyophilization cycle and SVP is less than 50 particles per mL for particles 10 μm or larger, and less than 5 particles per mL for particles 25 μm or larger.

Embodiment 74. A method of reducing low molecular weight species (LMW) in an rAAV product after lyophilizing the product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   storing said lyophilized composition for a duration at a temperature between −20° C. and 8° C., such as a temperature of −20° C. to 0° C. or 0° C. to 8° C., wherein the lyophilized product is reconstituted and LMW species are less than 5% by weight compared to the same product without lyophilization as measured by analytical ultracentrifugation.

Embodiment 75. A method of reducing low molecular weight species (LMW) in an rAAV product after lyophilizing the product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; and
   storing said lyophilized composition for a duration at a temperature between −20° C. and 8° C., such as a temperature of −20° C. to 0° C. or 0° C. to 8° C., wherein the lyophilized product is reconstituted and LMW species are less than 5% by weight compared to the same product without lyophilization as measured by analytical ultracentrifugation.

Embodiment 76. A method of reducing high molecular weight species (HMW) in an rAAV product after lyophilizing the product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
   storing said lyophilized composition for a duration at a temperature between −20° C. and 8° C., such as a temperature of −20° C. to 0° C. or 0° C. to 8° C., wherein the lyophilized product is reconstituted and HMW species increase by less than 3% by weight compared to the same product without lyophilization as measured by analytical ultracentrifugation.

Embodiment 77. A method of reducing high molecular weight (HMW) in an rAAV product after lyophilizing the product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; and
   storing said lyophilized composition for a duration at a temperature between −20° C. and 8° C., such as a temperature of −20° C. to 0° C. or 0° C. to 8° C., wherein the lyophilized product is reconstituted and HMW species increase by less than 3% by weight compared to the same product without lyophilization as measured by analytical ultracentrifugation.

Embodiment 78. The method of Embodiments 57-77, wherein the rAAV comprises a capsid derived from one or more AAVs selected from the group consisting of AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1 (VP1, SEQ ID NO: 1, or VP2, SEQ ID NO: 2, or VP3, SEQ ID NO: 3), DJ, DJ8, NP59, Anc-80, and variants thereof.

Embodiment 79. The method of any one of Embodiments 57-78, wherein the rAAV comprises a transgene that encodes a polypeptide, or a nucleic acid selected from the group consisting of a siRNA, an antisense molecule, miRNA, a ribozyme and a shRNA.

Embodiment 80. The method of Embodiment 79, wherein the rAAV comprises the transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

Embodiment 81. The method of Embodiment 80, wherein the rAAV comprises the transgene that encodes GAA (acid alpha-glucosidase).

Embodiment 82. The method of Embodiment 80, wherein the rAAV comprises the transgene that encodes ATP7B (copper transporting ATPase2).

Embodiment 83. The method of Embodiment 80, wherein the rAAV comprises the transgene that encodes alpha galactosidase A (GLA).

Embodiment 84. The method of Embodiment 80, wherein the rAAV comprises the transgene that encodes ASS1 (arginosuccinate synthase).

Embodiment 85. The method of Embodiment 80, wherein the rAAV comprises the transgene that encodes beta-glucocerebrosidase.

Embodiment 86. The method of Embodiment 80, wherein the rAAV comprises the transgene that encodes beta-hexosaminidase A.

Embodiment 87. The method of Embodiment 80, wherein the rAAV comprises the transgene that encodes SERPING1 (C1 protease inhibitor or C1 esterase inhibitor).

Embodiment 88. The method of Embodiment 80, wherein the rAAV comprises the transgene that encodes glucose-6-phosphatase.

Embodiment 89. The method of Embodiment 80, wherein the rAAV comprises the transgene that encodes CFTR (cystic fibrosis transmembrane regulator protein).

Embodiment 90. The method of Embodiment 80, wherein the rAAV comprises the transgene that encodes a blood coagulation (clotting) factor (e.g., Factor XIII, Factor IX, Factor VIII, Factor X, Factor VII, Factor VIIa, protein C).

Embodiment 91. The formulation and method of any of Embodiments 1-90, wherein the drug product is an rAAV, and the rAAV comprises a capsid comprises VP1 comprising the nucleotide sequence of SEQ ID NO: 1. In a further embodiment the rAAV capsid further comprises VP2 comprising the nucleotide sequence of SEQ ID NO: 2. In preferred embodiment, the rAAV capsid comprises VP1 comprising the nucleotide sequence of SEQ ID NO: 1, VP2 comprising the nucleotide sequence of SEQ ID NO: 2 and VP3 comprising the nucleotide sequence of SEQ ID NO: 3.

### ADDITIONAL ASPECTS AND EMBODIMENTS

Additional aspects, embodiments, and examples of combinations thereof include the following:

1. An recombinant adeno-associated virus (rAAV) formulation comprising the components:
a) about 1×10⁹ to about 1×10¹⁵ vg/mL rAAV;
b) an ionic strength of about 40 mM to about 90 mM;
c) a buffering agent;
d) 0.0005-0.05% by weight one or more non-ionic surfactants;
e) 1-10% by weight one or more sugars; and
f) water,
g) at a pH of about 5.0 to about 7.5.

2. The rAAV formulation of 1, wherein said formulation comprises the components:
a) 20 mM to 30 mM Na-Acetate;
b) 10 mM to 50 mM NaCl;
c) 1 mM MgCl₂ to 9.5 mM MgCl₂
d) 3% to 8% sucrose;
e) 0.0005% to 0.05% Poloxamer 188 (P188); and
f) water;
   wherein said pH is 5.0 to 6.0.

3. The rAAV formulation of 2, wherein said formulation comprises the components:
a) about 25 mM Na-Acetate;
b) about 25 mM NaCl;
c) about 5 mM MgCl₂
d) about 5.4% sucrose;
e) 0.01% P188; and
f) water;
   wherein said pH is about 5.2.

4. The rAAV formulation of 1, wherein said formulation comprises the components:
a) 5 mM to 20 mM Tris;
b) 50 mM to 90 mM NaCl;
c) 3% to 8% by weight sucrose;
d) 0.001% to 0.01% by weight Poloxamer 188 (P188); and
e) water;
   wherein said pH is 7.1 to 7.5.

5. The rAAV formulation of 4, wherein said formulation comprises the components:
a) about 10 mM Tris;
b) about 75 mM NaCl;
c) about 5% sucrose;
d) about 0.005% P188; and
e) water;
   wherein said pH is about 7.3.

6. The rAAV formulation of 1, wherein said formulation comprises the components:
a) 5 mM to 20 mM Na-citrate;
b) 50 mM to 90 mM NaCl;
c) 5% to 9% by weight sucrose;
d) 0.0005% to 0.01% by weight Poloxamer 188 (P188); and
e) water;
   wherein said pH is 5.5 to 6.5.

7. The rAAV formulation of 6, wherein said formulation comprises the components:
a) about 10 mM Na-citrate;
b) about 75 mM NaCl;
c) about 7% by weight sucrose;
d) about 0.001% P188; and
e) water;
   wherein said pH is about 6.0.

8. The rAAV formulation of any one of 1-7, wherein said formulation consists essentially of said components.

9. The rAAV formulation of any one of 1-7, wherein said formulation consists of said components.

10. The rAAV formulation of any one of 1-9, wherein the rAAV comprises a capsid comprising a VP1 comprising the sequence of SEQ ID NO: 1, a VP2 comprising the sequence of SEQ ID NO: 2 and a VP3 comprising the sequence of SEQ ID NO: 3.

11. The rAAV formulation of any one of 1-10, wherein the rAAV is provided at about 1×10¹⁰ to about 1×10¹⁴ vg/mL

12. The rAAV formulation of 11, wherein the rAAV is provided at about 1×10¹³ to about 4×10¹³ vg/mL.

13. The rAAV formulation of any one of 1-12, wherein the rAAV comprises a recombinant viral nucleic acid encoding GAA or GLA.

14. A lyophilized recombinant adeno-associated virus (rAAV) formulation made by a process comprising lyophilizing the rAAV formulation of any one of 1-13.

### EXAMPLES

The following examples are merely illustrative of the presently disclosed subject matter and should not be considered as limitations in any way.

### Example 1: Long Term Stability of rAAV in a Low-Ionic Strength Formulation

### A. Materials

Sodium acetate trihydrate, sodium chloride, magnesium chloride hexahydrate salts were procured from Fisher Scientific (Waltham, MA). Sodium phosphate dibasic and monobasic salts were from J. T. Baker (Radnor, PA) and sucrose was from Pfanstiehl (Waukegan, IL). Kolliphor® P188 BIO (Pluronic®F68) was procured from BASF (Ludwigshafen, Germany). AAV capsids of RHM4-1 were prepared as described in U.S. Pat. No. 9,840,719 (VP1 of SEQ ID NO: 1, VP2 of SEQ ID NO: 2 and VP3 of SEQ ID NO: 3), which is incorporated herein by reference. Multiple AAV vectors of the RHM4-1 capsid were prepared, each with a different transgene. One rAAV was produced containing a transgene encoding acid alpha-glucosidase (GAA) as described in US20210222141, the content of which is incorporated herein by reference. This GAA containing vector is herein referred to as AAV-GAA. Another type of vector with the same capsid contained a transgene encoding α-galactosidase A (GLA) as described in WO 2022/155665, the content of which is incorporated herein by reference. This GLA containing vector is herein referred to as AAV-GLA.

### B. Preparation of Formulations

Formulations were made by buffer exchange with the corresponding buffer in 24-well plates (molecular weight cut off (MWCO) of 100 kDa). All formulations were diluted to a target titer of 2×10¹³ vg/mL to 3×10¹³ vg/mL (2E13 vg/mL to 3E13 vg/mL) with the corresponding buffers and surfactants followed by final filtration with 0.22 um syringe filters (EMD Millipore, Burlington, MA). The final formulations labelled as F1 (formulation 1), F2 (formulation 2) and F3 (formulation 3) (Table 1) were filled into volumes in the range of 1.3-3 mL in sterile 5 mL CZ vials (West Pharmaceuticals, Upper Darby, PA), sealed and each put in stability chambers corresponding to conditions including −80° C., −20° C., −40° C., 5° C. and 25° C. At each time point from time 0 (referred to as T0) to 2 weeks (2 w), 2 months (2 m) and 6 months (6 m) given in Table 2, samples were pulled out from the stability study condition and aliquoted to 2 mL sterile CZ vials (West Pharmaceuticals, Upper Darby, PA) for testing the Critical Quality Attributes (CQAs) by various assays (given below).

TABLE 1
Formulations Used for Current Study and Formulation Components

| Formulation | Composition of formulation matrix |
|---|---|
| F1 | 25 mM Na Acetate, 25 mM NaCl, 5 mM MgCl₂, pH |
| | 5.2, 5.4% Sucrose, 0.01% Poloxamer 188 |
| F2 | 10 mM Na Phosphate, 180 mM NaCl, pH 7.3, 0.001% |
| | Poloxamer 188 |
| F3 | 10 mM Na Phosphate, 60 mM NaCl, 3% Sucrose, pH |
| | 7.3, 0.001% Poloxamer 188 |

TABLE 2
Time Points and Conditions of the Study

| | |
|---|---|
| Study Condition | Time points |

| | | |
|---|---|---|
| T0 | | Time 0 (frozen at −80° C.) |
| 25° | C. | 2 weeks and 2 months |
| −80° | C. | 2 months |
| −40° | C. | 2 months and 6 months |
| −20° | C. | 2 months and 6 months |
| 5° | C. | 2 months and 6 months |

### C. Visual Inspection

All samples were visually inspected under a light meter for the evaluation of appearance and to identify the presence of visible particles.

All samples of F1 (low pH and low ionic strength), F2 (conventional pH and conventional ionic strength), F3 (moderate ionic strength and conventional pH) at all study conditions including T0, 25° C. —2 weeks and 2 months, −80° C.—2 months, −20° C.—2 months and 6 months, −40° C.—2 months and 6 months, 5° C.—2 months and 6 months were found to be clear and colorless solutions with no visible particles.

### D. Glass Transition of the Freeze Concentrate (Tg′)

The glass transition temperatures of the freeze concentrate (Tg′) of all T0 formulation samples were determined by the method of Low temperature differential scanning calorimetry (LT-DSC) USP (United States Pharmacopeial Convention)<891>: Thermal Analysis was followed for LT-DSC using TA Instruments Q2000 with 9.8-18.6 mg of sample solution at the rate of 10° C./minute. The evolution or uptake of heat for the sample during the steps of cooling and warming during the thermal event reflected the energy differences which was recorded as measurements.

The glass transitions of the freeze concentrate (Tg′) analyzed by LT-DSC are given in Table 3. The Tg′ for F1 and F3 were noted to be −38.85° C. and −41.96° C. respectively. Tg′ could not be picked up for platform formulation (F2) as seen in the table, however it has been historically reported as about −65° C. The glass transition temperature for F1 was higher than that for F2. It is beneficial to store biologics below glass transition for enhanced stability with minimized mobility. Increased Tg′ also enables storage at higher temperatures other than −80° C., which is favorable and highly desirable considering the ease of supply chain practices including handling/shipping at higher temperatures. It is also amenable as an adoptable approach since it maximizes the sterility assurance (low temperature storage can compromise container closure systems, as −80° C. is generally below the glass transition of plastics and rubber due to which plastic generally becomes brittle at these low temperatures).

TABLE 3
Glass Transition Measurements of Formulations

Glass Transition

| Sample | Onset ° C. | Inflection point ° C. | Heat capacity |
|---|---|---|---|
| F1 | −39.73 | −38.85 | 0.15 |
| F3 | −42.71 | −41.96 | 0.11 |

### E. Vector Genome Titer by qPCR

The qPCR assay was performed to measure the vector genome copy numbers of all samples. The samples were first incubated with DNAse for 15 minutes at room temperature to digest the non-encapsulated vector genomic DNA. The DNAse reaction was stopped and samples were then incubated at 95° C. to open the capsid and then diluted to be within the range for standards based on estimated titer. Titer recoveries (%) were calculated based on titer measurements and comparisons to T0 values.

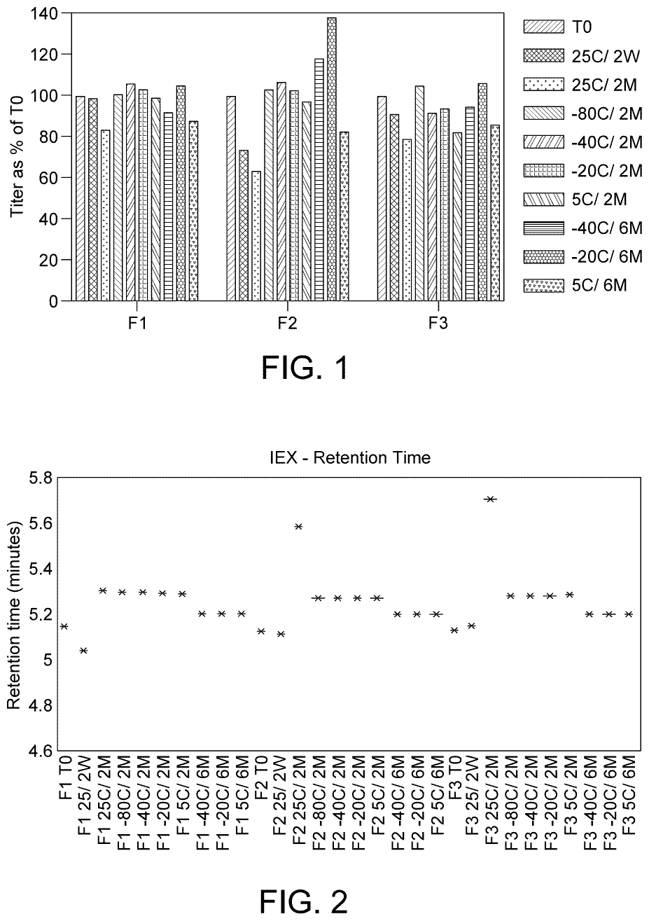

FIG. 1 and Table 4 shows the titer recoveries of all samples relative to T0 titers. The vector titer recovery was noted to be retained for F1 at all storage conditions including 5° C., −20° C., −40° C., −80° C. for longer time points including 2-6 months (87-107% genome recovery). The data are indicative of an extrapolated shelf life of >1.5 years at 2-8° C. if common acceptance criterion of 60-140% is used. Similarly, shelf life of >2 years for all frozen temperatures including −20° C. is supported. Vector titer recovery for F3 was also retained at all storage conditions including 5° C., −20° C., −40° C., −80° C. for longer time points including 2-6 months (86-107% genome recovery). However, in the F2 formulation (high ionic strength), the titer recovery dropped to ˜73.7% and 62.52% for 25° C.-2 weeks and 2 months, respectively, while samples from all other conditions exhibited higher titer recoveries. High ionic strength formulations (i.e. where NaCl>150 mM) are commonly used in rAAV preparations and previous reports indicated retention of recovery and stability for such formulations (see, Wright, J. Frasier, et al., “Identification of factors that contribute to recombinant AAV2 particle aggregation and methods to prevent its occurrence during vector purification and formulation”, Molecular Therapy, (2005) 12:1, Rodrigues, Gerard A., et al., “Pharmaceutical development of AAV-based gene therapy products for the eye”, Pharmaceutical Research, (2019) 36:29, and Srivastava, Arvind, et al. “Manufacturing challenges and rational formulation development for AAV viral vectors”, Journal of Pharmaceutical Sciences 110.7 (2021): 2609-2624). High ionic strength formulations are used for commercial marketed AAV products (see, e.g., ZOLGENSMA® package insert; LUXTURNA® package insert). It is shown here that, compared to conventional, high ionic strength formulations (e.g. F2), titer recovery is improved and maintained within the storage time of the study with moderate ionic strength formulations (50-95 mM; 60 mM F3). Titer recovery was best improved and maintained with a low ionic strength formulation (<50 mM; 25 mM F1) at low pH (<7). Titer recovery would also be maintained when extrapolated beyond 1.5 years.

TABLE 4
Vector Recoveries (%) For All Samples Calculated With Respect to T0 Titers

| | Titer recovery (% of T0) | | | | | | | | | |
|---|---|---|---|---|---|---|---|---|---|---|
| | | 25° C./ | 25° C./ | −80° C./ | −40° C./ | −20° C./ | 5° C./ | −40° C./ | −20° C./ | 5° C./ |
| Sample | T0 | 2 w | 2 m | 2 m | 2 m | 2 m | 2 m | 6 m | 6 m | 6 m |
| F1 | 100 | 98.80 | 83.40 | 100.99 | 106.39 | 103.31 | 99.32 | 92.24 | 105.26 | 87.85 |
| F2 | 100 | 73.70 | 62.52 | 103.05 | 106.31 | 102.60 | 97.22 | 118.42 | 137.67 | 82.69 |
| F3 | 100 | 91.12 | 79.28 | 104.83 | 91.74 | 93.82 | 82.47 | 94.96 | 106.27 | 85.77 |

Note:
w = weeks,
m = months

### F. Ion Exchange Chromatography (IEX) for % Full Capsids

The Ion Exchange Chromatography (IEX) assay was performed using standard methods and UV at 280 nm. The rAAV samples were predominantly pure of full rAAV, where the empty to full ratio was 3:7 or less. Samples retrieved for all formulation at all stability conditions were reported to be enriched for full particles only. The retention time was noted to undergo a shift for F2 and F3 formulations at 25° C. for 2 months (FIG. 2), indicating the possibility of deamidation, which was further confirmed by peptide mapping data. However, the retention time shift and deamidation was not observed for F1 samples across all study conditions. Deamidation is inversely correlated to efficacy and transduction of rAAV and hence highly undesirable (Giles, April R., et al. “Deamidation of amino acids on the surface of adeno-associated virus capsids leads to charge heterogeneity and altered vector function.” Molecular Therapy.2018. 26 (12): 2848-2862).

### G. SEC-MALS for Aggregation

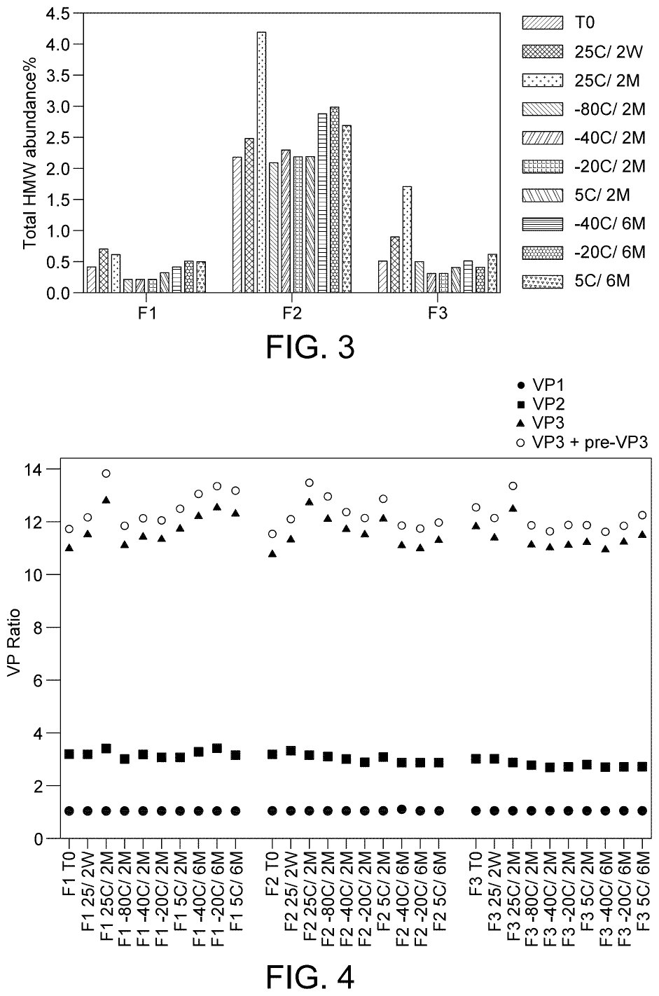

The monomer molecular weight, hydrodynamic radius, and concentration of empty, full, and total capsids was tested with size exclusion chromatography. Abundance of monomer and high molecular weight (HMW) species were equal to their percent of the total integrated ultraviolet signal at 280 nm. SEC MALS data revealed, as illustrated in FIG. 3, that the abundance of HMW species % indicating impurities and degradants are lesser for the low pH and low ionic strength formulation, F1, and for the moderately low ionic strength, F3, formulation relative to conventional pH and ionic strength, F2, formulation. This result indicates better quality and stability for F1 and F3 over F2. For F2, HMW % is relatively high for all stability conditions.

### H. CE-SDS for Capsid Protein Purity

Samples containing rAAV were denatured with sodium dodecyl sulfate (SDS) and beta-mercaptoethanol (BME) at 70° C. The denatured samples were subjected to capillary electrophoresis and separated proteins were detected by absorbance at 220 nm.

The VP protein ratios were not significantly impacted for all formulations under all stability conditions as seen in FIG. 4. Hence capsid protein purity was retained across all formulations and not negatively impacted by lower pH and/or ionic strengths.

### I. Dynamic Light Scattering (DLS) for Size

DLS was performed to study the intensity and volume particle size distribution (PSD) and to determine the mean hydrodynamic size (Z-average). Polydispersity Index (PDI) was also measured, which serves as an indicator for aggregation.

Reported DLS data is shown in Table 5, which shows that the vector quality and stability was retained for low pH and/or low ionic strength F1 and F3 formulations under different conditions. The particle size was maintained around 24-30 nm for both F1 and F3 formulations. PDI values for F1 were slightly higher than that for F2 and F3, but it is contributed by the complex species within the buffer matrix (confirmed with buffer scans) and not due to vector aggregation.

TABLE 5
Particle size distribution data from DLS showing
rAAV size, mean hydrodynamic diameter, polydispersity
index values for all samples.

| Formu- | | From Volume PSD | | | |
|---|---|---|---|---|---|
| lation | | SIZE d · nm | VOL % | Z-AVG d · nm | PDI |
| F1 | T0 | 28.27 | 100 | 33.02 | 0.236 |
| | 25° C., 2 w | 24.74 | 99.8 | 48.35 | 0.668 |
| | 25° C., 2 m | 25.55 | 99.6 | 63.62 | 0.895 |
| | −80° C., 2 m | 23.3 | 100 | 34.59 | 0.282 |
| | −40° C., 2 m | 27.05 | 99.9 | 34.52 | 0.308 |
| | −20° C., 2 m | 28.53 | 99.9 | 34.33 | 0.284 |
| | 5° C., 2 m | 27.34 | 99.9 | 34.65 | 0.32 |
| | −40° C., 6 m | 26.82 | 100 | 32.58 | 0.252 |
| | −20° C., 6 m | 29.49 | 100 | 32.03 | 0.21 |
| | 5° C., 6 m | 28.83 | 99 | 33.99 | 0.285 |
| F2 | T0 | 23.32 | 100 | 24.17 | 0.1 |
| | 25° C., 2 w | 22.91 | 100 | 24.85 | 0.088 |
| | 25° C., 2 m | 22.67 | 100 | 25.16 | 0.104 |
| | −80° C., 2 m | 23.82 | 100 | 24.81 | 0.07 |
| | −40° C., 2 m | 21.92 | 100 | 25.18 | 0.155 |
| | −20° C., 2 m | 23.36 | 100 | 24.24 | 0.081 |
| | 5° C., 2 m | 24.1 | 100 | 24.51 | 0.01 |
| | −40° C., 6 m | 23.16 | 100 | 24.24 | 0.058 |
| | −20° C., 6 m | 23.69 | 100 | 24.41 | 0.041 |
| | 5° C., 6 m | 23.12 | 100 | 24.11 | 0.058 |
| F3 | T0 | 25.2 | 100 | 26.69 | 0.121 |
| | 25° C., 2 w | 25.64 | 100 | 26.22 | 0.115 |
| | 25° C., 2 m | 27.2 | 100 | 27.19 | 0.176 |
| | −80° C., 2 m | 24.42 | 100 | 25.89 | 0.104 |
| | −40° C., 2 m | 26.52 | 100 | 26.44 | 0.068 |
| | −20° C., 2 m | 23.35 | 100 | 25.72 | 0.144 |
| | 5° C., 2 m | 23.66 | 100 | 25.6 | 0.127 |
| | −40° C., 6 m | 25.27 | 100 | 27.66 | 0.144 |
| | −20° C., 6 m | 25.14 | 100 | 27.35 | 0.187 |
| | 5° C., 6 m | 24.83 | 100 | 27.7 | 0.146 |

Note:
m = months,
w = weeks

### J. Intrinsic Differential Scanning Fluorimetry (DSF) for Thermal Stability

Changes in the intrinsic fluorescence signals of tryptophan residues present in rAAV capsids upon thermal ramping were assessed to study the thermal unfolding of viral proteins. The data corresponding to the fluorescence intensity ratio 350/330 nm and the changes in the viral protein melting temperatures (Tm) of T0 samples of all formulations were measured to investigate their thermodynamic stability using 10 uL samples in replicates in sealed capillaries.

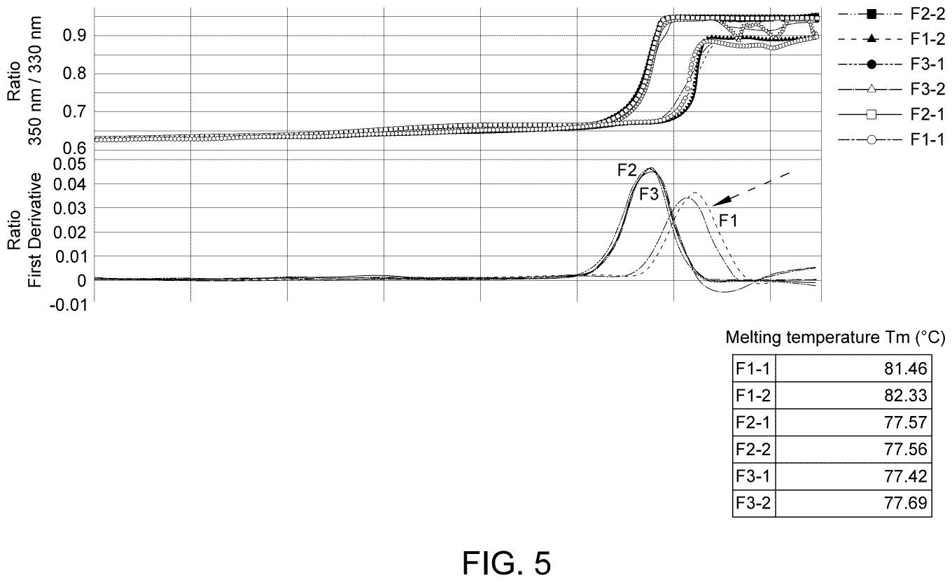

Intrinsic DSF data shown in FIG. 5 indicated that the melting point (Tm) of F1 was shifted by ˜5° C. relative to F2 and F3 showing the significant change in unfolding of viral proteins. F1 showed the highest Tm, thereby emphasizing superior thermodynamic or physical stability.

### K. Extrinsic Differential Scanning Fluorimetry (DSF) for Thermal Stability

The genome release from rAAV samples stained with a DNA binding dye, SYBR®Gold, upon thermal stress induction was detected by extrinsic DSF. Changes to the onset of DNA uncoating temperature were assessed for T0 samples to investigate the thermodynamic stability of rAAV formulations.

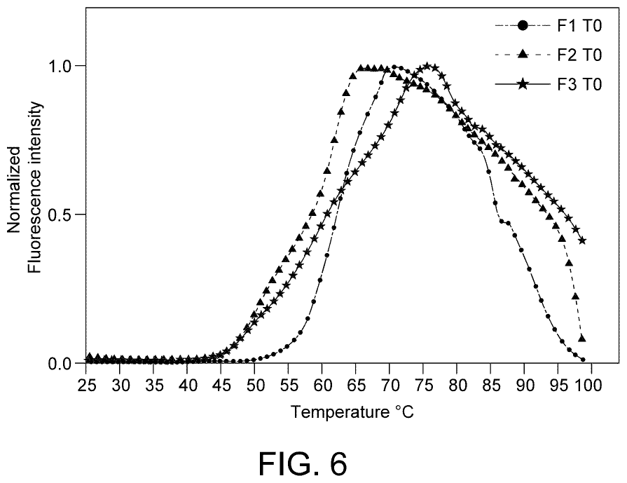

Extrinsic DSF assay using the DNA binding dye (SYBR®Gold) showed that the onset of DNA uncoating temperature for F1 was noted to shift by ˜5° C. compared to F2 and F3 (FIG. 6). The shift highlighted the higher thermodynamic stability of the F1 formulation (low ionic strength and low pH), and supports our conclusion that the capsid will be most compacted structurally around pH 5.5, which concurrently aligns with the isoelectric point.

### L. Light Obscuration for Sub-Visible Particle Assessment

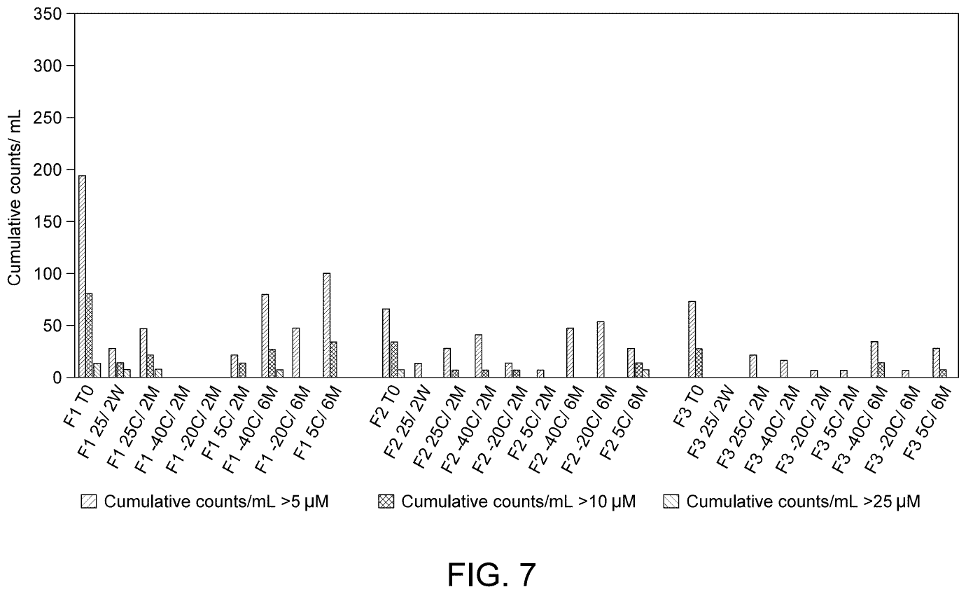

Subvisible particulates were measured with a light obscuration (HIAC) dependent particle counter. Particle measurements in different size buckets including >5 um, >10 μm and >25 um were noted as cumulative counts/mL. HIAC data of subvisible particle counts in all size ranges did not show any significant concerns (FIG. 7). All numbers passed USP (United States Pharmacopeial Convention)<787> criteria for allowable counts of subvisible particles in therapeutic protein injections.

### M. Peptide Mapping for Post-Translational Modifications (PTM)

Proteins in RHM4-1 capsids were denatured, disulfides were reduced to free thiols and irreversibly alkylated. Aliquots of the sample were separately enzymatically digested by trypsin and Asp-N, respectively. The peptides of each sample generated from enzymatic digest were separated by reverse phase liquid chromatography and detected by mass spectrometry. Raw data was processed using a bioinformatics pipeline where the sequence coverage was mapped by comparison between identified peptides and theoretical peptides of RHM4-1 capsid proteins. Each peptide was identified using intact molecular mass and sequence specific fragment ion data. Levels of PTM including deamidation and oxidation were determined.

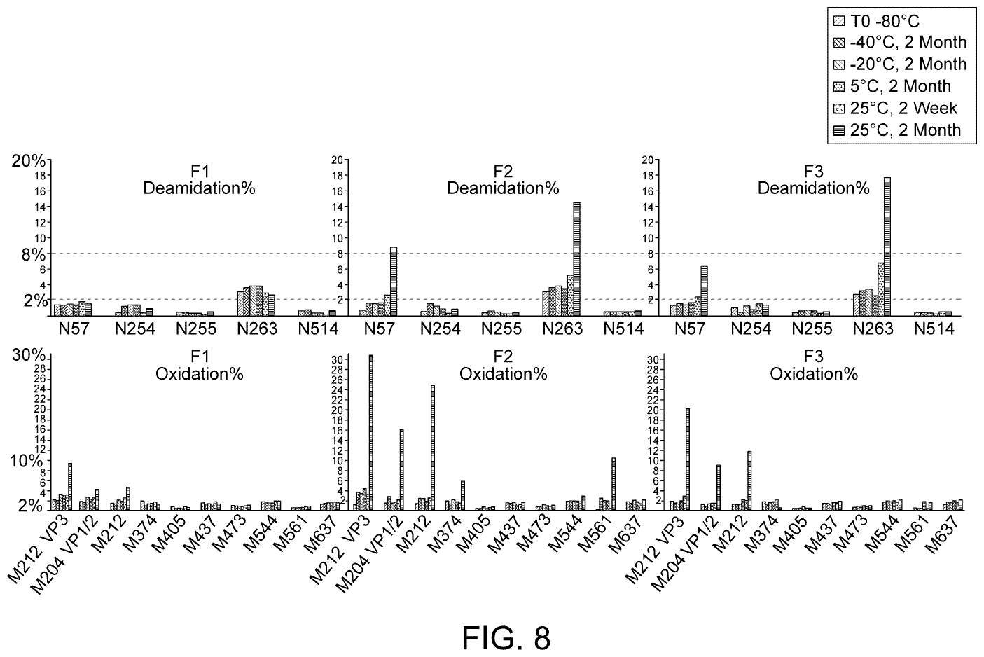

As seen in FIG. 8, F1 did not show any considerable levels of deamidation compared to F2 and F3, which showed higher levels particularly for 25° C.-2 week and 2 month samples (30-35%) in contrast to F1 (2.5%). Significant oxidation was noted for 25° C.-2 month stressed samples of F2 and F3 (20-30%) in contrast to F1 (9%).

### N. Potency by Cell-Based Assay

Samples with an engineered AAV vector encoding GAA (AAV-GAA) as described in US20210222141 were tested in a potency assay that measures GAA activity in cells transduced with GAA vector. Cells were plated in 96-well plates and transduced with GAA vector reference standard (RS), positive control sample (PC), and test article(s) (TA). On the third day after transduction, enzymatic activity (cleavage to release a fluorophore) of secreted GAA by the cells was measured spectrophotometrically using a fluorescence microplate reader. The fluorescence for each reference standard and test sample dilution was plotted against vector genome concentration, and the dose-response curves were fitted by linear regression software. The line for test sample was compared to that of the reference standard using slope ratio analysis, and relative potency of sample is reported as the percent of reference standard (% RP). Time zero (T0) samples stored at −80° C. for 6 months served as control samples for all formulations.

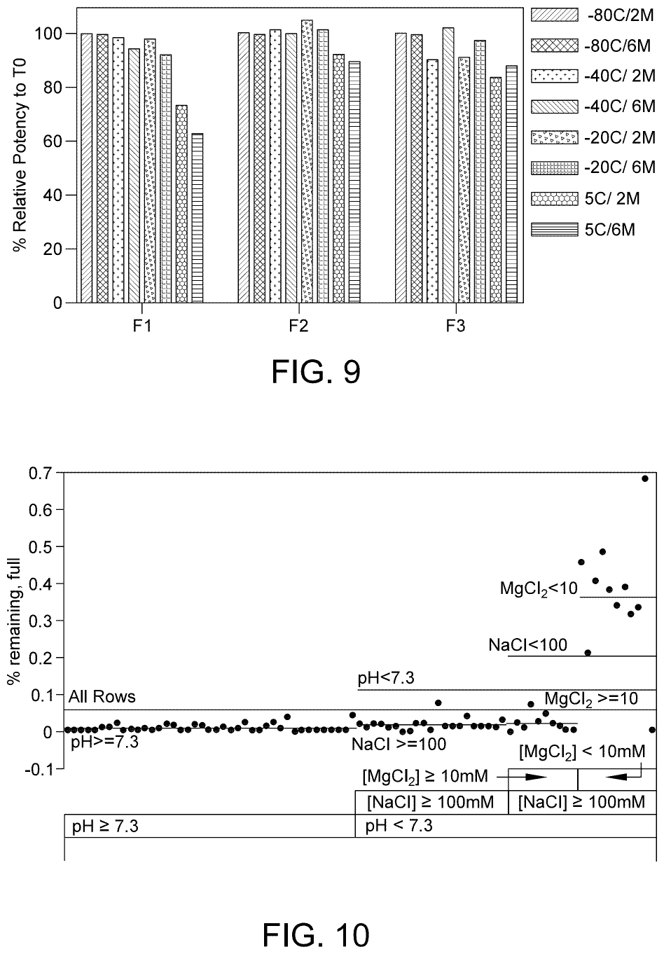

The formulation buffers were not observed to affect the cell viability. Potency data in FIG. 9 showed that F1 retained high relative potency for conditions including−20° C., −40° C., and −80° C. for 2 and 6 months, which also indicates the potential to store rAAV in F1 formulation at these temperatures for a shelf life of least 2 years. Formulations F2 and F3 retained high relative potency for 5° C., −20° C., −40° C., −80° C. The moderate ionic strength formulation F3 is expected to retain potency when and if stored for over 2 years at 2-8° C. (extrapolated from current data trending).

### O. Summary

The overall findings of the long-term stability study confirms that a low ionic strength, sucrose-based, surfactant containing, low pH formulation (F1) showed the best performance for most CQAs (i.e. had the best CQA profile). Favorable data with F1 including retention of titer and potency, less degradation, less aggregation, less impurities, and no potential of deamidation, as compared to the F2 and F3 formulations, also supports an extended shelf life at higher temperatures including-20° C. and −40° C. (relative to −80° C.) with the F1 formulation. Additionally, the presence of sucrose and the Tg′ data highlight the sugar's stabilizing effect and support the improved storage at frozen temperatures and amenability for lyophilization.

Those in the field understand that no single CQA is determinative, rather the CQA profile of each formulation will indicate which formulation is best for the particular purpose or product. Here, the low ionic strength and low pH formulation, F1, has the best CQA profile compared to the conventional and generally recognized higher physiological pH (7.3) and ionic strength (>150 mM NaCl) formulations for long term frozen storage and thawing of rAAV products. The addition of surfactant is also expected to minimize the potential adsorption losses to manufacturing and administration components thus ensuring high and enhanced dose accuracy.

### Example 2: Optimized Excipients to Reduce Degradation During Short-Term Stability

### A. RAAV Solution Preparation

Various matrix solutions were prepared with a variety of buffers, amino acid, salt, detergent, and sugar additives. Engineered rAAV products, as described above in Example 1, were prepared into the matrices by either dilution or buffer exchange. rAAV solutions were then exposed to short-term stress via controlled exposure to temperature (25-35° C.) for between 1-30 days.

### B. Forced Degradation in Liquid Phase

Loss of genome titer is a hallmark degradation mechanism of rAAV that reduces therapeutic effectiveness and increases immunogenicity. Short-term storage at elevated temperatures accelerates this degradation process relative to low storage temperatures, enabling assessment of key conditions to enhance stability for liquid storage in experimentally feasible timeframes.

rAAV were diluted into matrices of varied pH, ionic strength, and additional additives in 96-well microtiter plates. The solutions were sealed and incubated at between 30-35° C. to apply short-term stress. After exposing the rAAV solutions to stress, the solutions were cooled to 4° C. to minimize further changes, and analyzed to assess changes in titer using ion exchange-high performance liquid chromatography (IEX-HPLC).

Examples of changes in rAAV genome titer during forced degradation are shown in FIG. 10 for rAAV and analyzed using a partitioning coefficient model. Titer changes are measured by relative changes in the area of an intact full rAAV vector by HPLC. This analysis identifies three preferred conditions for preserving vector titer during incubation: pH<7.3, a concentration of NaCl<100 mM, and a concentration of MgCl₂<10 mM. Any of these attributes reduces titer loss during rAAV exposure. In certain embodiments, the combination of all three attributes in F1 may be preferred to best reduce the loss of rAAV titer.

A summary of statistical analysis to identify key parameters given in Table 6 supports these findings. The effect of the condition (pH, ionic strength (shown by sodium chloride concentration), magnesium, arginine, and histidine) on the prevention of titer loss is determined using Analysis of Variance (ANOVA). The effect is generally determined as “strong” or “moderate” if the effect probability (p) is below 0.01 or between 0.01-0.1, respectively. Conditions that improve titer loss include pH below 7.0, low concentrations of salt species (NaCl at a concentration<100 mM, MgCl₂ at a concentration <10 mM), and the addition of arginine and histidine. Importance was determined via analysis with ANOVA and Partition models. The preferred range indicates ranges that are preferred for a given attribute.

TABLE 6
Example of key excipients from forced degradation screening.

| Condition/Ingredient | Prevention of Titer Loss | Preferred Range | |
|---|---|---|---|
| pH | Strong | pH ≤ 6.5 | |
| Ionic Strength | Strong | 10-50 | mM |
| MgCl₂ | Strong | ≤5 | mM |
| Arginine | Medium | ≥20 | mM |
| Histidine | Medium | ≥20 | mM |

### C. Subvisible Particulate Formation During Freeze-Thaw

Freezing and thawing processes (F-T) during viral production are a common source of viral aggregation and subvisible particulate formation (SVP), increasing drug product immunogenicity and patient risk. Predictable particulate formation is required for freeze-thaw processes across key manufacturing scales and variation in product composition across batches, including pooling during downstream manufacturing, frozen product storage, and freezing for lyophilization. The impact of F-T on SVP formation in rAAV product (as described above) was studied by diluting the rAAV product into matrices with varied buffer, surfactant, salt, and other excipients. Over 100 experimental conditions were tested, with strategic variation of parameters to enable creation of a statistical model to describe the SVP formation. Solutions were sealed, exposed to F-T at controlled rates of temperature change, and monitored for generated SVP formation. SVP size and abundance were then fit to an empirical statistical model to understand the general behaviors observed across tested conditions.

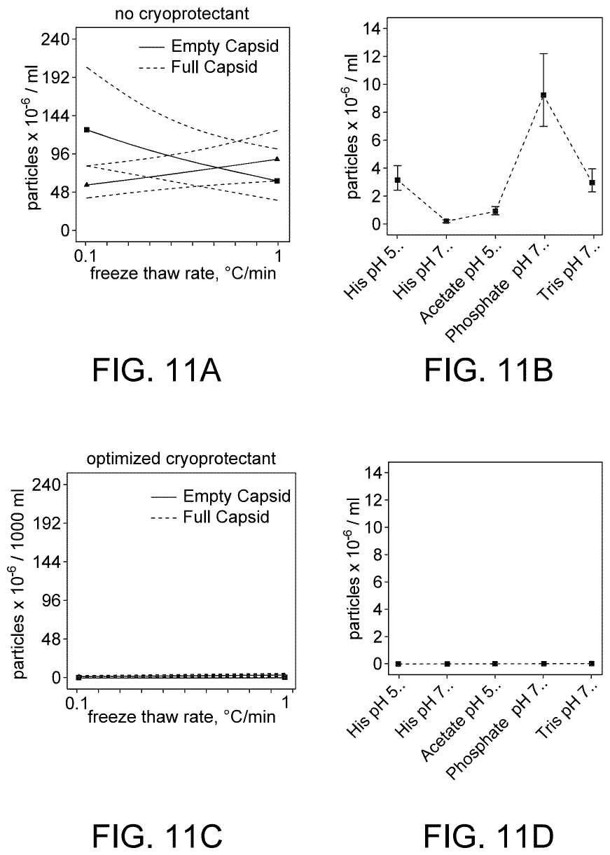

The statistical model for SVP formation in unoptimized matrices is shown in FIGS. 11A and 11B. The number and size of SVPs are highly dependent on F-T rate, empty rAAV content, and buffering species. Within the field, each of these parameters are difficult to control in production processes. Empty rAAV content refers to the proportion of empty particles (AAV that do not contain the complete genome) in the sample. It is known that empty rAAV content can vary widely across batches, with between 90% full to greater than 80% empty across batches and projects within a given serotype. Freeze-thaw rates can vary by more than 10-fold with changes in container size and freezing or thawing temperatures. Buffer species are often highly restricted by compatibility with key processing steps and/or patient administration routes. Dependence of SVP formation on such processing and production conditions may lead to undesirable and unpredictable SVP level in drug products that may vary with processing step, processing scale, storage temperature, batch-batch product composition variation, and route of administration to patients.

SVP formation with improved and predictable characteristics is shown in FIGS. 11C and 11D. The addition of sugar and surfactant is important to reduce overall SVP formation, as well to eliminate independent SVP formation on F-T rate, empty rAAV content, and buffers species at both neutral and acidic pH. The improved SVP formation is gained with the range of 0.001-0.05% surfactant, or more preferably 0.01-0.05%. Improved SVP formation is also seen for addition of sugars to 1-6%, or more preferably 2-4%. Statistical analysis of the effects to support these findings is shown in Table 7.

TABLE 7
Summary of matrix and excipient effects identified in Freeze-Thaw forced degradation.

| Condition | Effect on SVP Number | Preferred Direction or Range |
|---|---|---|
| Buffer: acetate, histidine, citrate | Strong effect in absence of | Acetate, histidine, preferred (in |
| (pH 5.2) | sucrose (sugar) and Poloxamer | absence of sugar and surfactant |
| | 188 (surfactant) | only). |
| Buffer: histidine, tris (pH 7-8) | Strong effect in absence of | Histidine, tris, preferred (in |
| | sucrose (sugar) and Poloxamer | absence of sugar and surfactant |
| | 188 (surfactant) | only). |
| Poloxamer 188 | Strong | 0.01-0.05, particularly if using |
| | | phosphate |
| Sucrose | Strong | 1-4%, particularly if using |
| | | phosphate |
| Poloxamer 188 and Sucrose | Strong | >0.01% Poloxamer 188 and >3% |
| combined | | sucrose preferred |
| Ionic Strength | Moderate | <100 mM preferred |

Together, these forced-degradation studies probe key effects of ionic strength, buffer species, pH, surfactant, sugar, vector purity (e.g., empty capsid percentage), and freeze-thaw rate on overall rAAV stability. Together, they identify the following: (a) moderate ionic strength can mitigate loss of both vector genome and vector aggregation; (b) both mildly acidic pH (5-6.5) and neutral pH (6.5-7.5) can stabilize rAAV; (c) the combination of non-ionic surfactant (with >0.005% v/v preferred) and sugar (>1.5% preferred) produces freezing properties where common buffering species are all equivalent and particle aggregation is not impacted by freezing rates or empty capsid concentration. Based on the results from Example 2 F4 and F5 formulations (F4 (Formulation 4)=10 mM Tris, 75 mM NaCl, 5% Sucrose, 0.005% Poloxamer 188, pH 7.3; F5 (Formulation 5)=10 mM Citrate, 75 mM NaCl, 7% sucrose, 0.001% Poloxamer, pH 6.0) were developed. Formulation amenable to lyophilization conditions include (a) moderate ionic strength of 50-95 mM, (b) pH of 5-7.5, (c) non-volatile buffer that can buffer in the range of pH 5-7.5, and minimum sugar concentration sufficient to provide for a stable lyophilized cake.

### Example 3: Long Term Stability of AAV-GLA in a Low-Ionic Strength Formulation

### A. Formulation Composition and Stability Study of AAV-GLA

A vector of the RHM4-1 capsid was prepared (as shown in U.S. Pat. No. 9,840,719 with VP1 of SEQ ID NO: 1, VP2 of SEQ ID NO: 2 and VP3 of SEQ ID NO: 3) containing a transgene encoding α-galactosidase A (GLA), (AAV-GLA), (as described in WO 2022/155665), was formulated in formulation F1 (25 mM sodium acetate, 25 mM NaCl, 5 mM MgCl₂, 5.4% Sucrose, 0.01% Kolliphor P188, pH 5.2) as a sterile, single dose, preservative-free aqueous solution in a 5 mL CZ vial (Cat number: 19550209, West Pharmaceutical Services).

AAV-GLA in formulation F1 was monitored for long-term stability at storage conditions including −80° C.±10° C. for 3, 6, 9 and 12 months, −20° C.±10° C. for 6, 12 months and 5° C.±3° C. for 1, 3 and 6 months. The study also evaluated the stability of material at accelerated stability conditions such as 25° C./60% RH for 2 weeks, and 1 month.

### B. PH of AAV-GLA During Storage

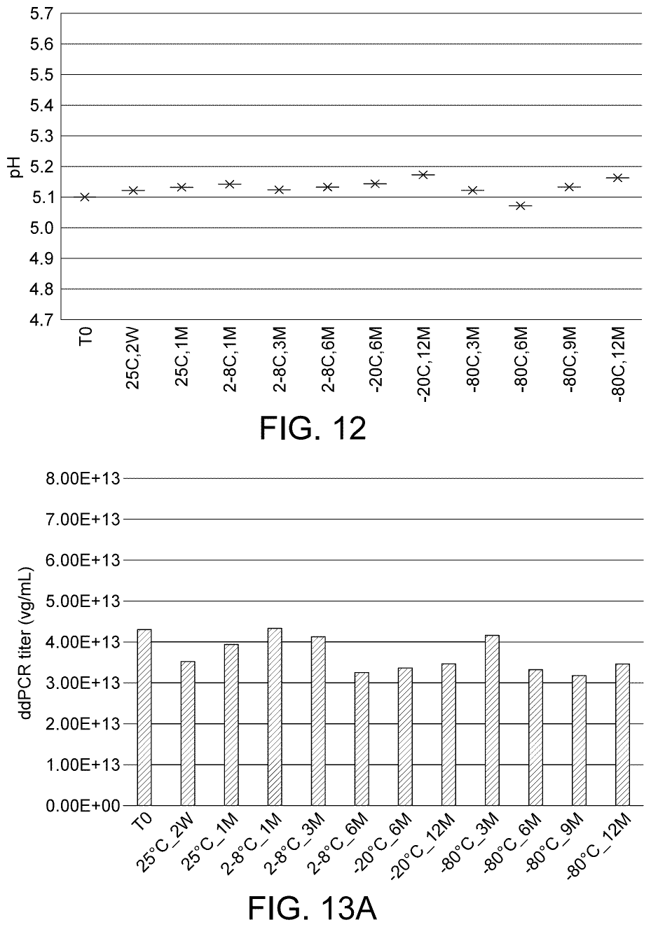

The pH of AAV-GLA during storage was measured using potentiometry. The analytical procedure was performed in accordance with the compendial procedures USP <791> and Ph. Eur. 2.2.3. The measured values after storage (FIG. 12) are consistent across all conditions and time points compared to T0, within the intended specification for pH.

### C. Vector Genome Titer by ddPCR

Viral genome titer of AAV-GLA in Formulation F1 during storage was measured using droplet digital polymerase chain reaction (ddPCR). Samples were added to a ddPCR solution containing transgene specific primers and fluorescent probe. Each sample was divided into uniform nanoliter-sized droplets, with target and background DNA distributed randomly into the droplets during the partitioning process. The droplets were transferred to a 96-well plate for PCR amplification where a segment of the transgene was amplified. A droplet reader reads each droplet to determine the fraction of positive droplets. The fraction of droplet positives was used to calculate the copy number concentration of target DNA. The vector genome concentration result for each test article and control were reported as vector genome copies per mL of product (vg/mL).

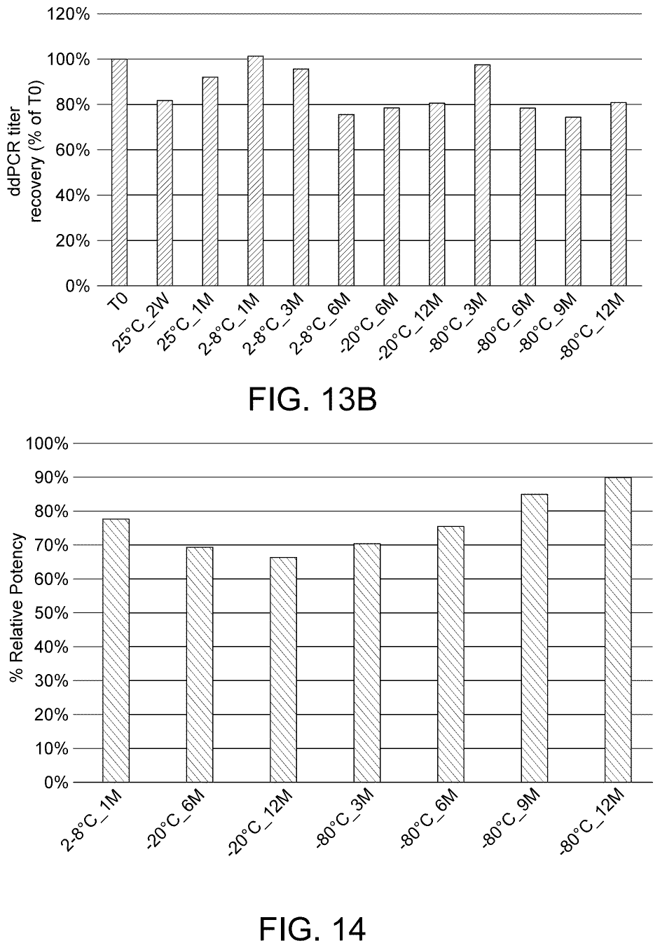

Vector titer and recovery (FIG. 13) shows the titer recoveries of all samples relative to T0 titers. The genome titer recovery was noted to be retained at −80° C. target storage condition for time points such as 3 (97%), 6 (78%), 9 (74%) and 12 (80%) months and therefore showed no concerns. 2-8° C. long term storage up to 6 months and −20° C. storage up to 12 months also showed higher recoveries (75-101%). It is therefore shown here that titer recovery is maintained at greater than 70% for up to samples up to 12 months.

### D. In-Vitro Potency

Cell-based potency was designed to reflect the mechanism of action of the α-Gal A protein in relation to the reference standard. Potency was measured by assessing enzymatic activity due to expression of α-GalA from AAV-GLA transduced reporter cells, with enzymatic activity assessed using a fluorogenic reporter.

Potency (FIG. 14) was retained within at least 65% across all samples at −20° C., −80° C., and 1 month and 2-8° C. Losses of up to 35% are within typical variation for cell-based potency assays without consistent decline for the −20° C. and −80° C. storage temperature. Together these results indicate suitability for storage between −20° C. and −80° C. for at least 1 year.

### E. In-Vitro Infectivity

Cell-based infectivity testing assessed the in vitro infectious activity of AAV-GLA. This cell-based method involved co-infecting AAV packaging cells which carry the AAV rep and cap genes with AAV-GLA and Ad5. The readout incorporated 50% endpoint determination of vector DNA by ddPCR using the same primer and probe reagents as the vector genome concentration ddPCR method. The data analysis and infectious titer calculations were based on the Kärber method. The data were reported as vector-particle concentration to infectious vector titer ratio.

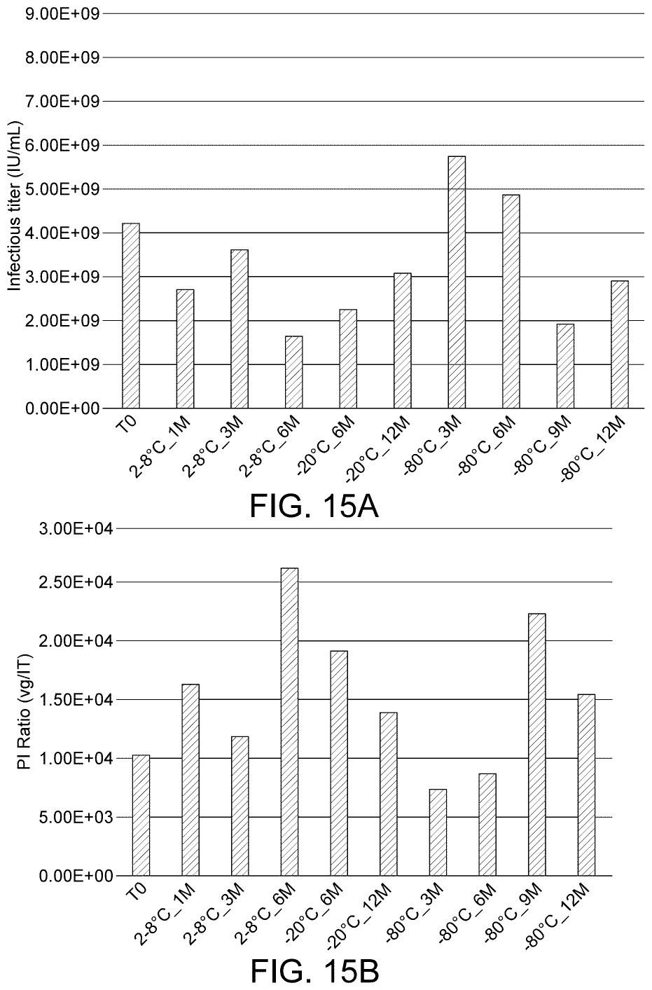

PI ratios (vector-particle concentration to infectious vector titer ratio, FIG. 15A) and infectious titers (FIG. 15B) exhibit variation typical of in-vitro cell-based assays. However behavior agrees with in-vitro cell based potency (FIG. 14) with no consistent decreasing infectivity. Importantly, infectious titer was found to be retained at 2-8° C. for 6 months and −20° C., −80° C. for all time points up to 12 months.

### F. Capsid Concentration and Empty: Full Ratio by UV Absorbance

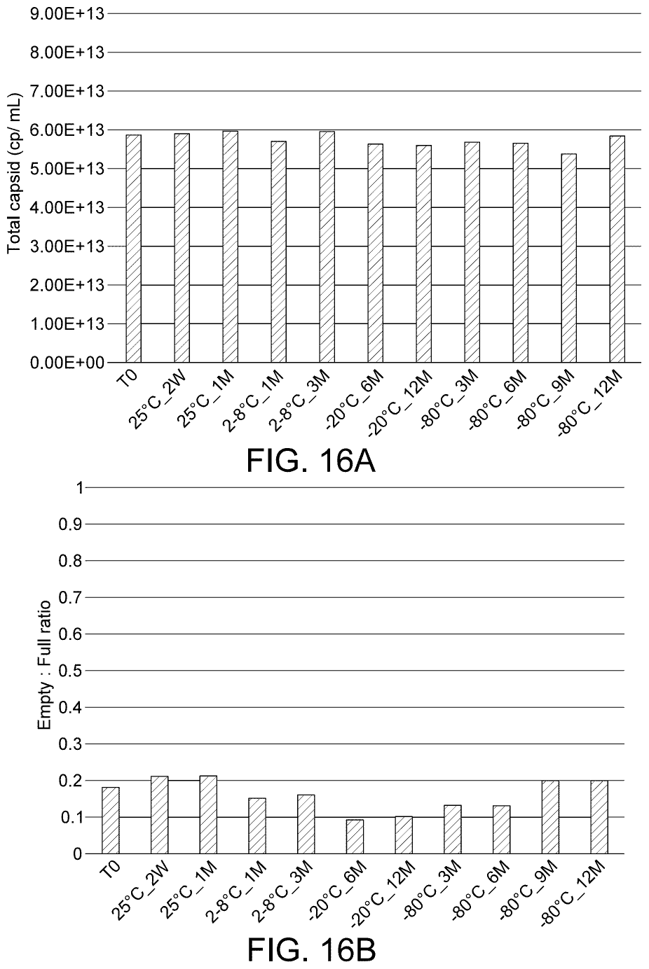

Absorbance at 280 nm and 260 nm were used to assess the total capsid and the ratio of empty capsid particles to full capsid particles (E: F) in AAV-GLA preparations. For all measurements, total capsid (FIG. 16A) is consistent between 5.5-5.9×10¹³ capsids/mL. and E: F ratio (FIG. 16A) is ≤0.25 for all samples, with no effective change during 12 months of storage.

### G. Capsid Purity and Ratio by CE-SDS

The relative abundance and purity of viral capsid proteins was assessed using capillary electrophoresis with sodium dodecyl sulphate (CE-SDS). This measures the abundance of VP1, VP2, and VP3 viral proteins, a capsid protein variant (pre-VP3), as well as key additional peptide impurities and fragments. The AAV-GLA particles were reduced and denatured. The reduced capsid proteins were separated based on their size using capillary electrophoresis (CE) based on the electrophoretic mobility of the protein molecules. The stoichiometric ratio of capsid proteins and capsid protein purity were determined based on corrected peak areas of VP1, VP2, VP3, and the VP3 variant pre-VP3.

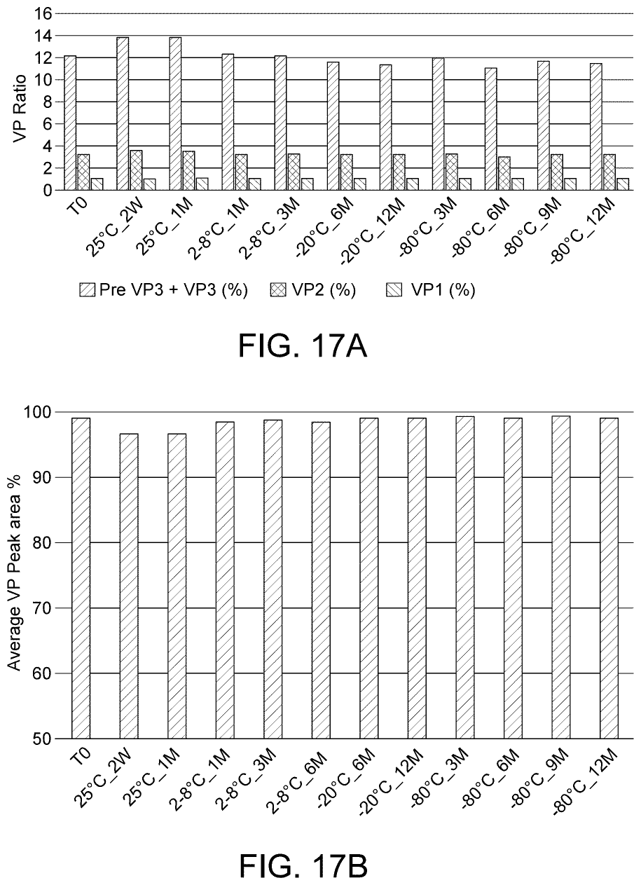

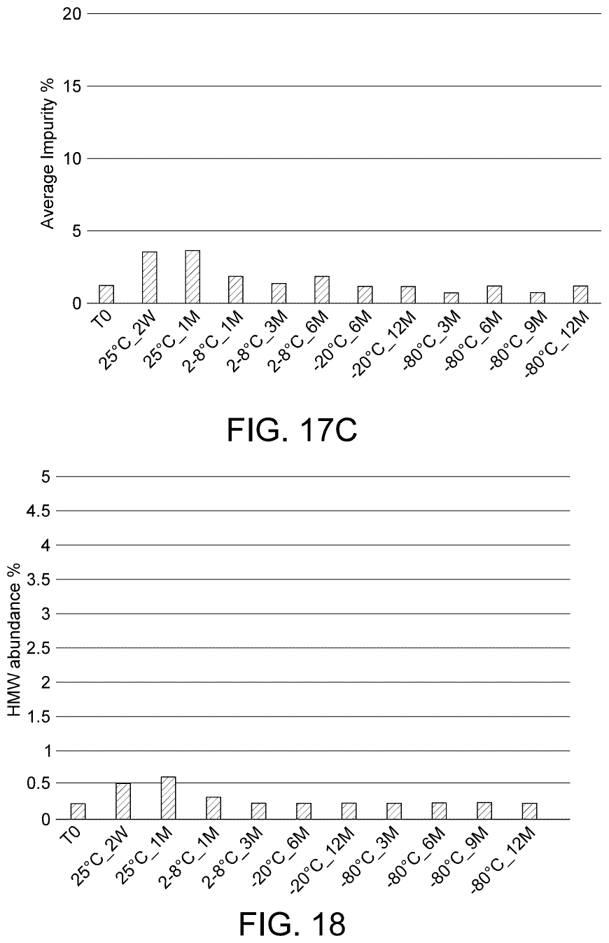

Relative ratios of VP proteins (FIG. 17A) vary within 20% of the measured T0 value, with no consistent increases or decreases during storage. Total purity of VP proteins (percent associated with VP1, VP2, and VP3, FIG. 17B) are above 98%, with a small decrease from T0 to 96% only associated with storage at 25° C. Similarly, the ratio of peptide impurities is consistently below 2% for all samples, except for 25° C. samples with a small increase to approximately 4%. FIG. 17C illustrates average impurity percent. Based on the data, protein purity was found to be retained for all storage conditions such as 2-8° C., −20° C. and −80° C. for at least 1 year.

### H. Percent Full Capsids by IEX

Capsid charge and charge variants were characterized with analytical anion exchange chromatography (AEX). Empty and full capsid particles were eluted from an analytical AEX column, with absorbance was monitored at 280 nm. The E: F ratio was determined by the ratio of empty capsid peak area to the full capsid peak area, after correction for extinction coefficient differences due to encapsidated DNA in full vs empty capsids.

TABLE 7
Ratio of empty:full capsids for AAV-GLA in formulation
F1 during long-term stability, measured by ion
exchange analytical chromatography.

| | Full Peak | |
|---|---|---|
| Sample | A260/280 | E:F Ratio |
| T0 | N/A | <LOD (E/F = 0.2) |
| 25° C._2 w | 1.3 | <LOD(E:F 0.2) |
| 25° C._1 M | 1.4 | <LOD(E:F 0.2) |
| 2-8° C._1 M | 1.3 | <LOD(E:F 0.2) |
| 2-8° C._3 M | 1.4 | <LOD(E:F 0.2) |
| 2-8° C._6 M | 1.4 | <LOD(E/F = 0.2) |
| −20° C._6 M | 1.4 | <LOD(E/F = 0.2) |
| −20° C._12 M | 1.3 | <LOD(E/F = 0.2) |
| −80° C._3 M | 1.3 | <LOD(E:F 0.2) |
| −80° C._6 M | 1.4 | <LOD(E/F = 0.2) |
| −80° C._9 M | 1.3 | <LOD(E/F = 0.2) |
| −80° C._12 M | 1.4 | <LOD(E/F = 0.2) |

IEX results are summarized in Table 7. Samples retrieved at all stability conditions were reported to be ≤0.2 (LOD) for E: F ratio and consistent A260/280 ratio of 1.3-1.4 for all samples through 12 months. There were no notable changes in the retention time for all samples; further implying no chances for deamidation (further supported by peptide mapping data).

### I. Aggregation by SEC-MALS

Oligomeric aggregates were assessed with analytical size exclusion with multi-angle light scattering (SEC-MALS), which separates species based on hydrodynamic volume. MALS detectors were used in-line to determine molecular weight and hydrodynamic radius in a single analysis. The relative abundance of high molecular weight species (FIG. 18) showed very similar monomer abundance levels (<1%) for all samples across the 1 year of storage.

### J. Sub-Visible Particle Assessment by Light Obscuration

Subvisible particulates were assessed using light obscuration (HIAC), using a low-volume 1 mL method. Particle measurements in different size buckets such as >5 μm, >10 μm and >25 μm were noted as cumulative counts/mL in Table 8. All conditions demonstrate very low levels of particulates, typically all at least 10-fold lower than the compendial limits for small-volume products. Furthermore, no apparent increase was observed for all samples within up to 1 year of storage.

TABLE 8
Sub visible particle counts measured by
HIAC for AAV-GLA in formulation F1.

| | Cumulative Counts/mL | | |
|---|---|---|---|
| Sample Name | >5 μM | >10 μM | >25 μM |
| T0 | 87 | 19 | 0 |
| 2-8° C._1 M | 0.00 | 0.00 | 0.00 |
| 2-8° C._6 M | 24.00 | 9.33 | 0.00 |
| −20° C._6 M | 108.00 | 18.67 | 1.33 |
| −80° C._6 M | 36.00 | 9.33 | 0.00 |
| −20° C._12 M | 34.67 | 1.33 | 0 |
| −80° C._12 M | 6.67 | 0 | 0 |

### K. Particle Size and Distribution by DLS

Particle size distribution and polydispersity were assessed with dynamic light scattering (DLS data (Table 9) demonstrate no discernable aggregation for all samples across 12 months, with main peak size (radius in nm) in the 25-32 nm range, the main peak polydispersity index (PDI)≤0.3, and the percent mass composition of monomer ≥98%.

TABLE 9
Aggregation of AAV-GLA after formulation in F1, storage, and reconstitution.
Aggregation assessed by DLS, with data reporting a cumulant fit to determine
an average molecular weight and polydispersity, and (b) a regularization
fit to estimate the relative abundance of monomer vs aggregates.

| | | | Regularization Fit | | | |
|---|---|---|---|---|---|---|
| | Cumulant Fit | | Monomer | Monomer | Monomer | HMW Mass |
| Sample Name | Z avg (nm) | PDI | Peak (nm) | PDI | Mass % | % |
| T0 | 30.1 ± 0.3 | 0.1 ± 0.0 | 31.9 ± 0.9 | 0.2 ± 0.1 | 98.1 ± 4.6 | 2.0 ± 4.6 |
| 25° C._2 w | 31.2 ± 0.3 | 0.1 ± 0.0 | 28.8 ± 0.6 | 0.2 ± 0.1 | 99.4 ± 0.9 | 0.6 ± 0.9 |
| 25° C._1 M | 28.3 ± 0.4 | 0.2 ± 0.0 | 26.0 ± 0.8 | 0.2 ± 0.1 | 98.4 ± 0.3 | 1.6 ± 0.3 |
| 2-8° C._1 M | 25.8 ± 0.1 | 0.1 ± 0.0 | 25.8 ± 0.5 | 0.1 ± 0.0 | 100.0 ± 0.0 | 0.0 ± 0.0 |
| 2-8° C._3 M | 25.5 ± 0.1 | 0.1 ± 0.0 | 27.2 ± 0.3 | 0.1 ± 0.0 | 100.0 ± 0.0 | 0.0 ± 0.0 |
| 2-8° C._6 M | 25.6 ± 0.2 | 0.1 ± 0.0 | 27.0 ± 0.2 | 0.1 ± 0.0 | 100.0 ± 0.0 | 0.0 ± 0.0 |
| −20° C._6 M | 25.9 ± 0.1 | 0.1 ± 0.0 | 27.4 ± 0.3 | 0.1 ± 0.0 | 100.0 ± 0.0 | 0.0 ± 0.0 |
| −20° C._12 M | 26.1 ± 0.1 | 0.1 ± 0.0 | 27.7 ± 0.2 | 0.14 ± 0.0 | 100.0 ± 0.0 | 0.0 ± 0.0 |
| −80° C._3 M | 26.0 ± 0.2 | 0.1 ± 0.0 | 27.4 ± 0.3 | 0.1 ± 0.0 | 99.9 ± 0.2 | 0.1 ± 0.2 |
| −80° C._6 M | 26.0 ± 0.1 | 0.1 ± 0.0 | 27.3 ± 0.3 | 0.1 ± 0.0 | 100.0 ± 0.0 | 0.0 ± 0.0 |
| −80° C._9 M | 25.8 ± 0.1 | 0.1 ± 0.0 | 27.4 ± 0.3 | 0.1 ± 0.0 | 100.0 ± 0.0 | 0.0 ± 0.0 |
| −80° C._12 M | 26.4 ± 0.2 | 0.1 ± 0.0 | 28.0 ± 0.2 | 0.15 ± 0.0 | 100.0 ± 0.0 | 0.0 ± 0.0 |

### L. Peptide Mapping for Post-Translational Modifications (PTM)

Chemical degradation of key peptides were analyzed with mass spectrometry with limited proteolysis. Samples of RHM4-1 capsid proteins were denatured, disulfides reduced to free thiols and then irreversibly alkylated. The sample was enzymatically digested, separated by reverse phase liquid chromatography, and detected by a mass spectrometer. Raw data was processed using a bioinformatics pipeline where the sequence coverage was mapped by comparison between identified peptides and theoretical peptides of RHM4-1 capsid proteins. Each peptide was identified using intact molecular mass and sequence specific fragment ion data, and analyzed for post translational modifications deamidation and oxidation were determined.

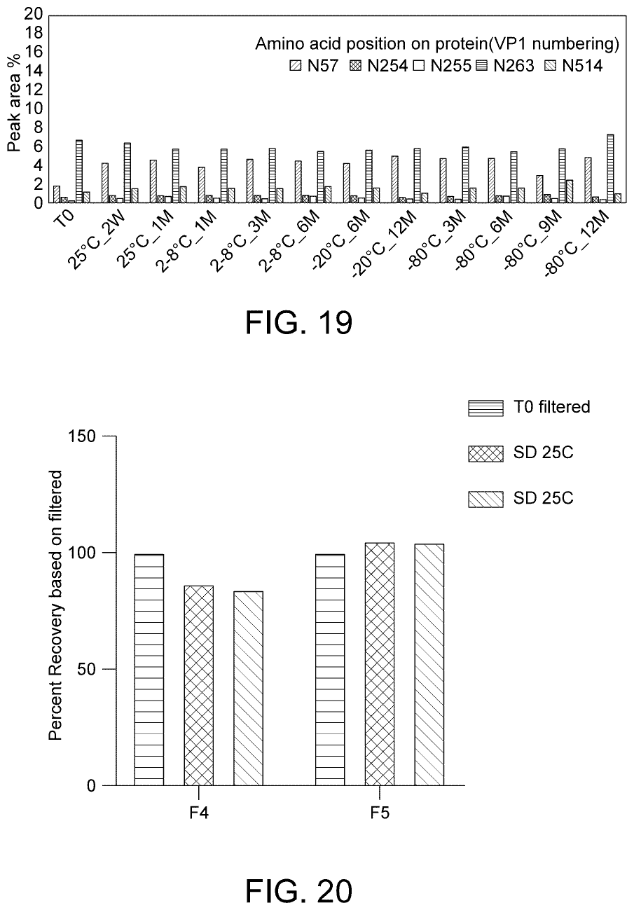

Deamidation levels (FIG. 19) were observed to be less than or equal to 8% across all asparagine residues for all samples from all conditions. Deamidation also shows no apparent increase for any residue.

### M. Summary

The overall findings of the long-term stability study confirms that a low ionic strength, sucrose-based, high Pluronic, low pH formulation demonstrated no change in analytical attributes over one year of monitoring at −20° C. Many attributes, including aggregation, particulate formation, and vector genome titer also displayed no changes at accelerated stability conditions (4° C. and 25° C.) for up to 6 months. Together, this indicates profound stability that is acceptable for at least 1 year between −20° C. and −80° C., and reasonably to at least 2 years.

### Example 4. Stability of Lyophilized AAV-GAA in a Moderate-Ionic Strength Formulation

### A. Stability after a Lyophilization Cycle

As described in Example 2, formulation development supported modifications to F1 to create moderate ionic strength formulations, F4 and F5 (described in Table 10.) Stability of AAV-GAA under a lyophilization cycle was tested in the moderate ionic strength formulations F4 and F5. rAAV solutions were prepared by buffer exchange of purified AAV-GAA into aqueous solutions and addition of key excipients, and filtration through 0.2 μm, and filled in glass vials. Solutions were frozen at −50° C., warmed to −35° C. for primary drying, and warmed to 25° C. for 30° C. for secondary drying. The lyophilized cake was then reconstituted in deionized water and tested for changes in critical quality attributes.

TABLE 10
Solutions in lyophilization cycle testing.

| Formulation name | Matrix |
|---|---|
| F4 | 10 mM Tris, 75 mM NaCl, 5% |
| | sucrose, 0.005% P188, pH 7.3 |
| F5 | 10 mM sodium citrate, 75 mM |
| | NaCl, 7% sucrose, 0.001% |
| | P188, pH 6.0 |

### B. Vector Recovery after Lyophilization

After the lyophilization cycle, the lyophilized cake was reconstituted and assayed for encapsulated genome titer to verify titer changes due to the lyophilization process. Vector genome titer was measured using quantitative polymerase chain reaction (qPCR). FIG. 20 depicts the titer before and after reconstitution for secondary drying at 25° C. versus 35° C. Titer was normalized relative to the titer before lyophilization (T0). AAV-GAA exhibits 15% titer in F4 and 5% increase in F5 (within experimental error of typical qPCR assays, indicating no degradation from the lyophilization cycle in either F4 or F5.

### C. Potency after Lyophilization

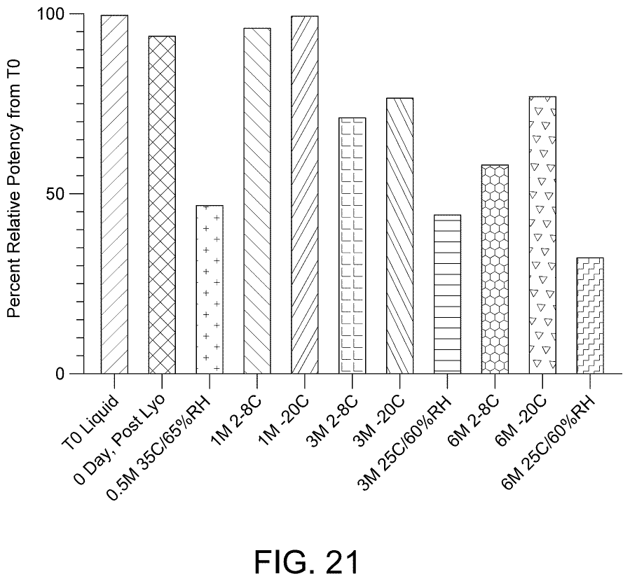

After the lyophilization cycle, the cake was reconstituted and assayed for in vitro cell potency to verify titer changes due to the lyophilization process. FIG. 21 depicts the relative cell potency after lyophilization, normalized to the potency before lyophilization. The vectors exhibit less than 10% relative potency loss, indicating acceptable degradation due to the lyophilization process in these solution matrices.

### D. High Molecular Weight after Lyophilization

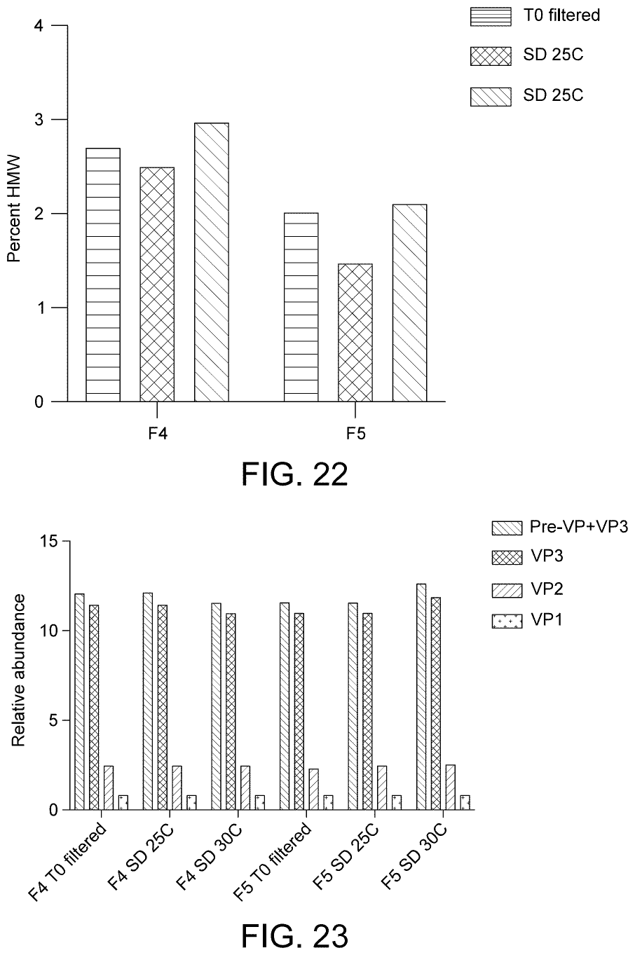

After the lyophilization cycle, the cake was reconstituted and assayed for high molecular species using SEC-MALS. FIG. 22 depicts the percentage of high-molecular weight species as determined by percent of area at 280 nm absorbance. All samples exhibit less than a 0.5% increase in high molecular weight species, indicating minimal impact of the lyophilization process on aggregate species.

### E. VP Protein Integrity after Lyophilization

After the lyophilization cycle, the cake was reconstituted and assayed for changes in the capsid proteins. FIG. 23 depicts the relative ratio of VP1, VP2, VP3, and pre-VP3 proteins. All ratios are normalized to the VP1 occupancy, and the pre-VP3 indicating a common degradant species formed from degradation of VP2 and VP3. F5 formulation with secondary drying at 30° C. indicates some fragmentation, with an approximately 15% increase in VP3 and pre-VP3. All other conditions show no major change from the T0.

### F. Long-Term Stability of a Lyophilized AAV-GAA Formulation F4

AAV-GAA in formulation F4 was lyophilized and monitored for long-term stability. rAAV solutions were prepared by buffer exchange of purified AAV-GAA vector, diluted to target titers of 1.5E13 vg/mL, sugar and surfactant were added from high concentration stocks, and the final solution was filtered through 0.22 μm filters. The prepared rAAV solutions were then lyophilized by freezing at −50° C., primary drying at −35° C., and secondary drying at 25° C. or 30° C. Vials were then stoppered under filtered nitrogen (NF) and sealed. Sealed vials were stored as described in Table 11. After the durations given in Table 11, samples were reconstituted and aliquoted into 2 mL sterile vials for testing the critical quality attributes by various assays.

TABLE 11
Conditions in long term stability study of
lyophilized rAAV-GAA in formulation F4.

| | Time Points |
|---|---|
| T0 Liquid | Time 0 (frozen at −80 C.) |
| T0 | Time 0 (lyophilized) |
| 25 C./60% RH | 3 months and 6 months |
| 35 C./65% RH | 2 weeks |

| | | |
|---|---|---|
| −20 | C. | 1 month, 3 months and 6 months |
| 2-8 | C. | 1 month, 3 months, 6 months, 12 months, 18 |
| | | months and 24 months |

### G. Vector Genome Titer by qPCR

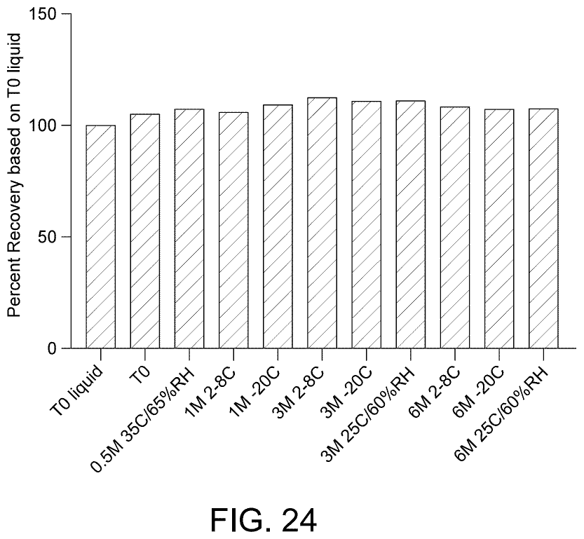

The titer of lyophilized AAV-GAA during storage (FIG. 24) is assessed with qPCR. Titer of all samples is equivalent or greater than the T0 control (no lyophilization) titer. The vector titer recovery was thus retained for all storage conditions including 35° C./65% RH, 25° C./60% RH, −20° C., and 2-8° C. for up to 6 months (100-114% genome recovery), with no appreciable decrease after up to 6 months of storage at room temperature.

### H. Potency by Cell-Based Assay

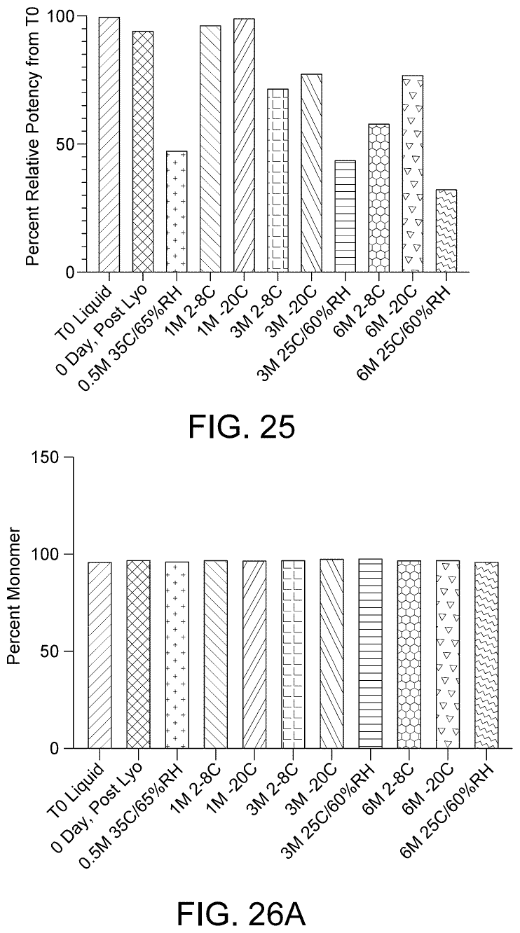

In-vitro cell-based potency (FIG. 25) assesses the functional activity of lyophilized AAV-GAA during storage. The lyophilized formulation retains a potency of at least 60% for conditions at 2-8° C. and −20° C. This is equivalent to the control T0, as in-vitro potency assays have large relative standard errors of at least 20%. Some decrease in potency is observed at 35 C/65% RH (approximately 50% decrease in 0.5 months) and 25° C./60% RH (52% after 3 months, 63% after 6 months). This decrease is acceptable for such accelerated stability conditions and at temperatures significantly above storage (−20° C. to 4° C.).

### I. Oligomeric Aggregation (SEC-MALS)

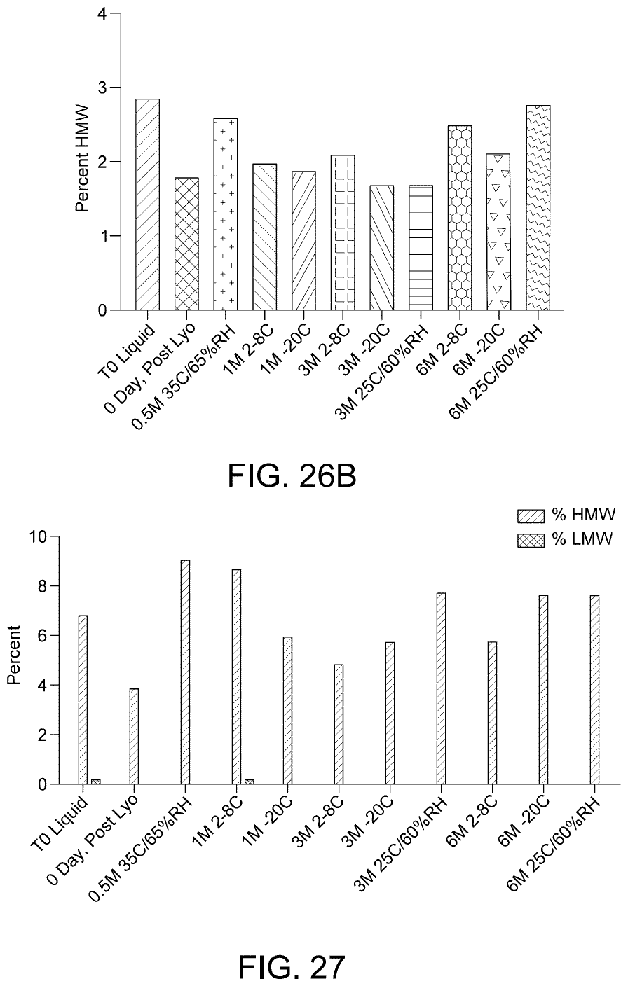

The formation of oligomeric high-molecular weight species (HMW) in lyophilized AAV-GAA were assessed with Size Exclusion Chromatography with multi-angle light scattering, and reported as the percentage of the total integrated UV signal at 280. As shown in FIGS. 26A to 26C, the relative abundance of were all between 2-3% for all samples before lyophilization and throughout storage, indicating no change in monomer state and oligomer aggregation across the study. From this, it is reasonable to extrapolate that storage at 4° C. would expect negligible HMW formation over at least 1 year.

### J. Fragmentation and Aggregation (Analytical Ultracentrifugation)

Analytical ultracentrifugation is a sensitive method for detection of high molecular weight species (HMW) and capsid fragments (low molecular weight species, LMW), key degradants that may form during storage. Reconstituted lyophilized AAV-GAA was assessed with AUC using a CsCl₂ gradient, monitoring of the sedimentation profile with absorbance at 230 nm, and modeling using the Lamm equation.

FIG. 27 shows HMW, partial, and LMW populations across storage. For both HMW and partials, all samples vary within 2% of the sample before lyophilization, indicating no effective change. LMW species were below the limit of detection for most samples (approximately 5%), indicating no fragmentation upon lyophilization or storage for at least 6 months. Storage at 4° C. would be expected to cause negligible change in LMW, HMW, and E: F over at least 1 year.

### K. Sub-Visible Particle Formation

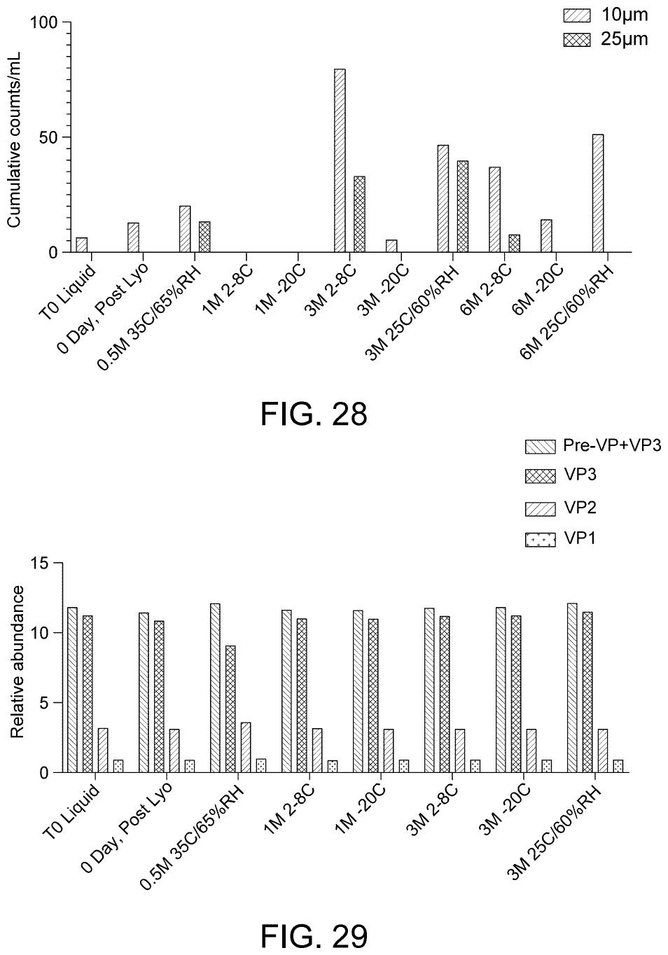

Subvisible particulates are a tightly regulated attribute for parenteral herapies. Subvisible particulates were measured by light obscuration using a 1 mL low volume method. Particle measurements are shown in FIG. 28 in different size bins of ≥10 μm and >25 μm, and noted as cumulative counts/mL. Measured levels are below 50 particles/mL for all samples, including the 35° C. and 25° C. accelerated stress conditions, all of which are over 100-fold below compendial guidelines in USP <787>.

### L. CE-SDS

The ratio of capsid viral proteins was assessed for reconstituted lyophilized AAV-GAA. Abundance of VP proteins was monitored with capillary electrophoresis under reduced conditions with SDS surfactant. VP ratios normalized to VP1 occupancy (FIG. 29) show no change for all storage conditions between −20° C. and 25° C. Relative to the pre-lyophilization condition, the 35° C./65% RH condition shows an approximately 10% increase in the Pre-vp3+vp3 abundance, indicating some fragmentation of the VP proteins at this condition. However, this change is acceptable for accelerated stability. All sample timepoints show no significant additional changes compared to T0 liquid (before lyophilization).

### M. Summary of Lyophilization Stability

Lyophilization cycle and long-term storage were tested for AAV-GAA. Across two medium ionic strength formulations F4 and F5, lyophilization cycle demonstrated minimal impact on rAAV titer, potency, and key attributes including capsid fragmentation, aggregation, and subvisible particles. Long term stability in formulation F4 demonstrated no change in titer, aggregation, capsid fragmentation, and VP protein fragmentation across 6 months and 4° C. In-vitro potency demonstrated variation within two standard deviations of assay error (40%) for storage up to 4° C. for 6 months, with only significant decreases only for accelerated stability conditions at 3 months. Thus, these moderate ionic strength formulations adequately stabilize rAAV during lyophilization and through storage at −20° C. to 4° C. for at least 1 year.

What is claimed is:

1. A stable formulation comprising a therapeutic drug product and:
a) 1-100 mM one or more salts;
b) a buffering agent;
c) 0.001-0.05% by weight one or more non-ionic surfactants;
d) 1-10% by weight one or more sugars; and
e) water,
f) optionally, one or more amino acids, and
wherein the composition has a pH of 4.5-7.5, and 60% or more of the drug product is recovered after the formulation is frozen and thawed.

2. The stable formulation of claim 1, wherein the buffering agent:
a) comprises a non-phosphate buffering agent;
b) is sodium acetate; and/or
c) is present in the formulation at a concentration of 1 mM to about 50 mM.

3. The stable formulation of claim 1, wherein the one or more salts:
a) includes 10-100 mM sodium chloride;
b) comprises a first salt and a second salt; and/or
c) comprises sodium chloride and magnesium chloride.

4. The stable formulation of claim 3, wherein the sodium chloride is present in the formulation at a concentration of about 25 mM, and/or the magnesium chloride is present in the formulation at a concentration of about 1 mM to about 10 mM, or is present at a concentration of about 5 mM.

5. The stable formulation of claim 1, wherein the one or more sugars comprises sucrose, and the sucrose content of the formulation is 5.4%.

6. The stable formulation of claim 1, wherein the non-ionic surfactant:
a) is present in the formulation at a concentration of about 0.01%;
b) is poloxamer 188, polysorbate 20, polysorbate 80, or poloxamer 407; and/or
c) is polysorbate selected from the group consisting of polysorbate 20 and polysorbate 80.

7. The stable formulation of claim 1, wherein the buffering agent is a phosphate buffering agent.

8. The stable formulation of claim 1, wherein the therapeutic drug product is:
a) a recombinant adeno-associated virus (rAAV); or
b) capable of increasing protein levels in a cell of GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USHIC (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

9. A stable formulation comprising:
i. (a) a recombinant adeno-associated virus (rAAV);
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5;
ii. (a) a recombinant adeno-associated virus (rAAV);
(b) 10 mM Tris;
(c) 75 mM sodium chloride;
(d) 0.005% by weight poloxamer 188;
(e) 5% by weight sucrose;
(f) water; and
(g) at a pH of 7.3; or
iii. (a) a recombinant adeno-associated virus (rAAV);
(b) 10 mM sodium citrate;
(c) 75 mM sodium chloride;
(d) 0.001% by weight poloxamer 188;
(e) 7% by weight sucrose;
(f) water; and
(g) at a pH of 6.0.

10. The stable formulation of claim 9, wherein:
(a) the rAAV comprises a capsid derived from one or more AAVs selected from the group consisting of AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1 (VP1, SEQ ID NO: 1, or VP2, SEQ ID NO: 2, or VP3, SEQ ID NO: 3), DJ, DJ8, NP59, Anc-80, and variants thereof;
(b) the rAAV comprises a transgene that encodes a polypeptide, or a nucleic acid selected from the group consisting of a siRNA, an antisense molecule, miRNA, a ribozyme and a shRNA;
(c) the rAAV comprises the transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USHIC (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof; or
(d) the rAAV comprises the transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), or a blood coagulation (clotting) factor (e.g., Factor XIII, Factor IX, Factor VIII, Factor X, Factor VII, Factor VIIa, protein C).

11. The stable formulation of claim 1 or 9, wherein after the composition is frozen for a duration at a temperature above −80° C. and up to −10° C.:
a) 60% or more of the therapeutic drug product or rAAV is recovered after thawing compared to the same product stored at −80° C. for same duration;
b) the drug product or rAAV exhibits at least 50% relative potency compared to the same product stored at −80° C. for the same duration;
c) the drug product or rAAV exhibits a glass transition temperature of freeze concentrate of −45° C. to −35° C., such as −45° C., −44° C., −43° C., −42° C., −41° C., −40° C., −39° C., −38° C., −37° C., −36° C. or −35° C.

12. The stable formulation of claim 1 or 9, wherein the composition is liquid or lyophilized.

13. A method of reducing degradation or retaining at least 50% relative potency of a therapeutic drug product after freeze-thawing said product, the method comprising preparing a composition comprising:
i. (a) the therapeutic drug product;
(b) 1-100 mM one or more salts;
(c) a buffering agent;
(d) 0.001-0.05% by weight one or more non-ionic surfactants;
(e) 3-10% by weight one or more sugars; and
(f) water,
(g) at a pH of 4.5 to 7.5; and
(h) optionally, one or more amino acids; or
ii. (a) the therapeutic product;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5; and
i) freezing said composition for a duration at a temperature above −80° C. and up to −10° C., such as a temperature of −70° C. to −15° C., −60° C. to −20° C., −50° C. to −30° C., or −45° C. to −35° C., wherein 60% or more of the therapeutic drug product is recovered after thawing compared to the same product stored at −80° C. for same duration; or
ii) freezing said composition for a duration at a temperature above −60° C. and up to −10° C., such as −60° C. to −10° C., −50° C. to −20° C., −45° C. to −25° C., or −30° C. to −40° C., wherein after thawing, said drug product exhibits at least 50% relative potency compared to the same product stored at −80° C. for the same duration.

14. A method of reducing degradation or retaining at least 50% relative potency of a therapeutic drug product, the method comprising preparing a composition comprising:
i. (a) the therapeutic product;
(b) 10 mM Tris;
(c) 75 mM sodium chloride;
(d) 0.005% by weight poloxamer 188;
(e) 5% by weight sucrose;
(f) water;
(g) at a pH of 7.3; or
ii. (a) the therapeutic product;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
i. lyophilizing said composition and storing for a duration at a temperature between −20° C. and 8° C., such as a temperature of −20° C. to 0° C. or 0° C. to 8° C., wherein 60% or more of the therapeutic drug product is recovered after reconstituting lyophilized composition compared to the same product stored at −80° C. without lyophilization; or
ii. lyophilizing said composition and storing for a duration at a temperature between −20° C. and 8° C., such as a temperature of −20° C. to 0° C. or 0° C. to 8° C., wherein said drug product exhibits at least 50% relative potency after reconstituting lyophilized composition compared to the same product stored at −80° C. without lyophilization.

15. The method of claim 13 or 14, wherein the therapeutic drug product is a recombinant adeno-associated virus (rAAV).

16. A method of reducing deamination of amino acids on capsid of an rAAV, the method comprising preparing a composition comprising:
i. (a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5;
ii. (a) the rAAV;
(b) 10 mM Tris;
(c) 75 mM sodium chloride;
(d) 0.005% by weight poloxamer 188;
(e) 5% by weight sucrose; and
(f) water; and
(g) at a pH of 7.3; or
iii. (a) the rAAV;
(b) 10 mM sodium citrate;
(c) 75 mM sodium chloride;
(d) 0.001% by weight poloxamer 188;
(e) 7% by weight sucrose; and
(f) water; and
(g) at a pH of 6.0.

17. A method of reducing degradation of viral proteins of an rAAV product after freezing and thawing the composition, the method comprising preparing a composition comprising:
(a) the rAAV;
(b) 25 mM sodium chloride;
(c) 5 mM magnesium chloride;
(d) 25 mM sodium acetate;
(e) 0.01% by weight poloxamer 188;
(f) 5.4% by weight sucrose; and
(g) water; and
(h) at a pH of 5 to 5.5; and
storing said frozen composition for a duration at a temperature between −80° C. and up to −10° C., such as a temperature of −70° C. to −15° C., −60° C. to −20° C., −50° C. to −30° C., or −45° C. to −35° C., wherein the change in the ratio VP1: VP3 is less than 20% in the thawed composition compared to before freezing said composition.

18. A method of reducing degradation of viral proteins of an rAAV product after lyophilizing the product, the method comprising preparing a composition comprising:
(a) the rAAV;
(b) 10 mM Tris;
(c) 75 mM sodium chloride;
(d) 0.005% by weight poloxamer 188;
(e) 5% by weight sucrose;
(f) water; and
(g) at a pH of 7.3; and
storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to before lyophilization.

19. A method of reducing degradation of viral proteins of an rAAV product after lyophilizing the product, the method comprising preparing a composition comprising:
(a) the rAAV;
(b) 10 mM sodium citrate;
(c) 75 mM sodium chloride;
(d) 0.001% by weight poloxamer 188;
(e) 7% by weight sucrose; and
(f) water;
(g) at a pH of 6.0; and
storing said lyophilized composition for a duration at a temperature between −20° C. and 8° C., such as a temperature of −20° C. to 0° C. or 0° C. to 8° C., wherein the change in the ratio VP1: VP3 is less than 20% in the reconstituted lyophilized product compared to before lyophilization.

20. The method of claim 13 or 14, wherein the rAAV comprises:
(a) a capsid derived from one or more AAVs selected from the group consisting of AAV1, AAV2, AAV3, AAV4, AAV5, AAV6, AAV7, AAV8, AAV9, AAV9.47, AAV9 (hu14), AAV10, AAV11, AAV12, Rh8, Rh10, Rh74, AAV3B, AAV-218, RHM4-1 (VP1, SEQ ID NO: 1, or VP2, SEQ ID NO: 2, or VP3, SEQ ID NO: 3), DJ, DJ8, NP59, Anc-80, and variants thereof;
(b) a transgene that encodes a polypeptide, or a nucleic acid selected from the group consisting of a siRNA, an antisense molecule, miRNA, a ribozyme and a shRNA; or
(c) a transgene that encodes GAA (acid alpha-glucosidase), ATP7B (copper transporting ATPase2), alpha galactosidase A (GLA), ASS1 (arginosuccinate synthase), beta-glucocerebrosidase, beta-hexosaminidase A, SERPING1 (C1 protease inhibitor or C1 esterase inhibitor), glucose-6-phosphatase, CFTR (cystic fibrosis transmembrane regulator protein), a blood coagulation (clotting) factor (e.g., Factor XIII, Factor IX, Factor VIII, Factor X, Factor VII, Factor VIIa, protein C), a gain of function blood coagulation factor, an antibody, retinal pigment epithelium-specific 65 kDa protein (RPE65), erythropoietin, LDL (low density lipoprotein) receptor, lipoprotein lipase (LPL), ornithine transcarbamylase (OTC), β-globin, α-globin, spectrin, α-antitrypsin, adenosine deaminase (ADA), a metal transporter (ATP7A or ATP7), sulfamidase, an enzyme involved in lysosomal storage disease (ARSA), hypoxanthine guanine phosphoribosyl transferase, β-25 glucocerebrosidase, sphingomyelinase, lysosomal hexosaminidase, branched-chain keto acid dehydrogenase, a hormone, a growth factor, insulin-like growth factor 1 or 2, platelet derived growth factor (PDGF), epidermal growth factor (EGF), nerve growth factor (NGF), neurotrophic factor-3 and -4, brain-derived neurotrophic factor (BDNF), glial cell line-derived growth factor (GDNF), transforming growth factor α and β, a cytokine, α-interferon, β-interferon, interferon-γ, interleukin-2 (IL-2), interleukin-4 (IL-4), interleukin 12 (IL-12), granulocyte-macrophage colony stimulating factor (GM-CSF), lymphotoxin (LT), a suicide gene product, herpes simplex virus thymidine kinase, cytosine deaminase (CD), diphtheria toxin (DT), cytochrome P450 (CYP), deoxycytidine kinase (DCK), tumor necrosis factor (TNF), a drug resistance protein, a tumor suppressor protein (e.g., p53, Rb, Wt-1, NF1, Von Hippel-Lindau (VHL), adenomatous polyposis coli (APC)), a peptide with immunomodulatory properties, a tolerogenic or immunogenic peptide or protein Tregitope or hCDR1 (Edratide), insulin, glucokinase (GCK), guanylate cyclase 2D (GUCY2D) (or Leber congenital amaurosis (LCA) associated with GUCY2D variants (GUCY2D-LCA)), Rab escort protein 1 (REP1) (choroideremia (CHM) encodes REP1), LCA 5 (Leber congenital amaurosis 5) (LCA-Lebercilin), ornithine ketoacid aminotransferase (gyrate atrophy), retinoschisin 1 (X-linked retinoschisis), USH1C (Usher's Syndrome 1C), X-linked retinitis pigmentosa GTPase (XLRP), MERTK (AR forms of RP: retinitis pigmentosa), DFNB1 (connexin 26 deafness), ACHM 2, 3 and 4 (achromatopsia), PKD-1+ or PKD-2 (polycystic kidney disease), TPP1 (tripeptidyl peptidase 1), CLN2 (Neuronal ceroid lipofuscinosis 2), a sulfatase, N-acetylglucosamine-1-phosphate transferase, cathepsin A, GM2-AP (ganglioside GM2 activator), NPC1 (NPC intracellular cholesterol transporter 1), VPC2, a sphingolipid activator protein, one or more zinc finger nucleases for genome editing, one or more donor sequences used as repair templates for genome editing, or variants thereof.

21. The method of claim 13 or 14, wherein the selected rAAV comprises:
(a) VP1, SEQ ID NO: 1, or VP2, SEQ ID NO: 2, or VP3, SEQ ID NO: 3.

22. The formulation of claim 1 or 9, wherein said drug product or said rAAV comprises a capsid comprising VP1 of SEQ ID NO 1, or VP2, SEQ ID NO: 2, or VP3, SEQ ID NO: 3.


SEQUENCE LISTING (3 sequences)

SEQ ID NO: 1 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 2 - protein, 601 aa, synthetic construct
TAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 3 - protein, 535 aa, synthetic construct
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL